SEQUENCE LISTING (201 sequences)

SEQ ID NO: 1 - DNA, 270 bp, synthetic construct
  source 1..270: mol_type = other DNA
ttaatgatgatgatgatgatgagcagatttagcaggtttcaatggagcacaatacatttccaatcttctcaaatcacaagatctaaaacaacattcatcaacaataccagtttgtggagctcttctagaagaagaaccataaccagttggtttattaaaataaaaacctctatcaccacaaacaaattgcaaagcatcaaccaattcagcaccacacaaagttcttggaccattagcaaacaaagaagacaatggcatagcaggaaacat

SEQ ID NO: 2 - DNA, 40 bp, synthetic construct
  source 1..40: mol_type = other DNA
gatttgacccttaatgatgatgatgatgatgtctcaattc

SEQ ID NO: 3 - DNA, 34 bp, synthetic construct
  source 1..34: mol_type = other DNA
ctaatctaagaattctgattctgaatgtccattg

SEQ ID NO: 4 - DNA, 39 bp, synthetic construct
  source 1..39: mol_type = other DNA
gaatcagaattcttagattagattgctatgctttctttc

SEQ ID NO: 5 - DNA, 43 bp, synthetic construct
  source 1..43: mol_type = other DNA
catcatcatcatcatcattaagggtcaaatcgttggtagatac

SEQ ID NO: 6 - DNA, 33 bp, synthetic construct
  source 1..33: mol_type = other DNA
ctaatctaagatggctgctggttctattactac

SEQ ID NO: 7 - DNA, 40 bp, synthetic construct
  source 1..40: mol_type = other DNA
gatttgacccttaatgatgatgatgatgatgagatttagc

SEQ ID NO: 8 - DNA, 38 bp, synthetic construct
  source 1..38: mol_type = other DNA
cagcagccatcttagattagattgctatgctttctttc

SEQ ID NO: 9 - DNA, 43 bp, synthetic construct; identical to SEQ ID NO: 5
  source 1..43: mol_type = other DNA

SEQ ID NO: 10 - DNA, 33 bp, synthetic construct
  source 1..33: mol_type = other DNA
ctaatctaagtctttgggttctttgactattgc

SEQ ID NO: 11 - DNA, 39 bp, synthetic construct
  source 1..39: mol_type = other DNA
gatttgacccttaatgatgatgatgatgatgagtaacag

SEQ ID NO: 12 - DNA, 39 bp, synthetic construct
  source 1..39: mol_type = other DNA
gaacccaaagacttagattagattgctatgctttctttc

SEQ ID NO: 13 - DNA, 43 bp, synthetic construct; identical to SEQ ID NO: 5
  source 1..43: mol_type = other DNA

SEQ ID NO: 14 - DNA, 31 bp, synthetic construct
  source 1..31: mol_type = other DNA
ctaatctaagatgtttcctgctatgccattg

SEQ ID NO: 15 - DNA, 36 bp, synthetic construct
  source 1..36: mol_type = other DNA
gatttgacccttaatgatgatgatgatgatgagcag

SEQ ID NO: 16 - DNA, 43 bp, synthetic construct; identical to SEQ ID NO: 5
  source 1..43: mol_type = other DNA

SEQ ID NO: 17 - DNA, 38 bp, synthetic construct
  source 1..38: mol_type = other DNA
caggaaacatcttagattagattgctatgctttctttc

SEQ ID NO: 18 - DNA, 52 bp, synthetic construct
  source 1..52: mol_type = other DNA
gaatcagaatttttgttttgtgtgtaaatttagtgaagtactgttttttgtg

SEQ ID NO: 19 - DNA, 45 bp, synthetic construct
  source 1..45: mol_type = other DNA
ctttgaaatggcgagtattgataatgagaaaccacaccgtggggc

SEQ ID NO: 20 - DNA, 42 bp, synthetic construct
  source 1..42: mol_type = other DNA
cacaaaacaaaaattctgattctgaatgtccattgtctcatg

SEQ ID NO: 21 - DNA, 45 bp, synthetic construct
  source 1..45: mol_type = other DNA
cggtgtggtttctcattatcaatactcgccatttcaaagaatacg

SEQ ID NO: 22 - DNA, 60 bp, synthetic construct
  source 1..60: mol_type = other DNA
gaatcagaatttgttttatatttgttgtaaaaagtagataattacttccttgatgatctg

SEQ ID NO: 23 - DNA, 47 bp, synthetic construct
  source 1..47: mol_type = other DNA
ggcgagtattgataatgagtgagtaaggaaagagtgaggaactatcg

SEQ ID NO: 24 - DNA, 44 bp, synthetic construct
  source 1..44: mol_type = other DNA
caaatataaaacaaattctgattctgaatgtccattgtctcatg

SEQ ID NO: 25 - DNA, 43 bp, synthetic construct
  source 1..43: mol_type = other DNA
ccttactcactcattatcaatactcgccatttcaaagaatacg

SEQ ID NO: 26 - DNA, 59 bp, synthetic construct
  source 1..59: mol_type = other DNA
gaatcagaatttttgaatatgtattacttggttatggttatatatgacaaaagaaaaag

SEQ ID NO: 27 - DNA, 51 bp, synthetic construct
  source 1..51: mol_type = other DNA
gtattgataatgaataacaatactgacagtactaaataattgcctacttgg

SEQ ID NO: 28 - DNA, 51 bp, synthetic construct
  source 1..51: mol_type = other DNA
ccaagtaatacatattcaaaaattctgattctgaatgtccattgtctcatg

SEQ ID NO: 29 - DNA, 52 bp, synthetic construct
  source 1..52: mol_type = other DNA
gtactgtcagtattgttattcattatcaatactcgccatttcaaagaatacg

SEQ ID NO: 30 - DNA, 53 bp, synthetic construct
  source 1..53: mol_type = other DNA
cagaatcagaatttttgtttgtttatgtgtgtttattcgaaactaagttcttg

SEQ ID NO: 31 - DNA, 52 bp, synthetic construct
  source 1..52: mol_type = other DNA
ccactagttctagaatccgataaaaaacacgctttttcagttcgagtttatc

SEQ ID NO: 32 - DNA, 45 bp, synthetic construct
  source 1..45: mol_type = other DNA
cataaacaaacaaaaattctgattctgaatgtccattgtctcatg

SEQ ID NO: 33 - DNA, 44 bp, synthetic construct
  source 1..44: mol_type = other DNA
gaaaaagcgtgttttttatcggattctagaactagtggatcccc

SEQ ID NO: 34 - DNA, 53 bp, synthetic construct
  source 1..53: mol_type = other DNA
gaatcagaattttttgagggaatattcaactgtttttttttatcatgttgatg

SEQ ID NO: 35 - DNA, 51 bp, synthetic construct
  source 1..51: mol_type = other DNA
ggcgagtattgataatgatttgccagcttactatccttcttgaaaatatgc

SEQ ID NO: 36 - DNA, 47 bp, synthetic construct
  source 1..47: mol_type = other DNA
gaatattccctcaaaaaattctgattctgaatgtccattgtctcatg

SEQ ID NO: 37 - DNA, 46 bp, synthetic construct
  source 1..46: mol_type = other DNA
gtaagctggcaaatcattatcaatactcgccatttcaaagaatacg

SEQ ID NO: 38 - DNA, 46 bp, synthetic construct
  source 1..46: mol_type = other DNA
cagaatcagaatttttttctattctgctgtttgctgtacctctttc

SEQ ID NO: 39 - DNA, 42 bp, synthetic construct
  source 1..42: mol_type = other DNA
ggcgagtattgataatgagtcattgtaccgcgaactttggac

SEQ ID NO: 40 - DNA, 58 bp, synthetic construct
  source 1..58: mol_type = other DNA
ggtacagcaaacagcagaatagaaaaaaattctgattctgaatgtccattgtctcatg

SEQ ID NO: 41 - DNA, 61 bp, synthetic construct
  source 1..61: mol_type = other DNA
ggtacaatgactcattatcaatactcgccatttcaaagaatacgtaaataattaatagtag

SEQ ID NO: 42 - DNA, 57 bp, synthetic construct
  source 1..57: mol_type = other DNA
cagaatcagaattgtttagttaattatagttcgttgaccgtatattctaaaaacaag

SEQ ID NO: 43 - DNA, 53 bp, synthetic construct
  source 1..53: mol_type = other DNA
gaaatggcgagtattgataatgaggggccgtatacttacatatagtagatgtc

SEQ ID NO: 44 - DNA, 47 bp, synthetic construct
  source 1..47: mol_type = other DNA
ctataattaactaaacaattctgattctgaatgtccattgtctcatg

SEQ ID NO: 45 - DNA, 45 bp, synthetic construct
  source 1..45: mol_type = other DNA
gtatacggcccctcattatcaatactcgccatttcaaagaatacg

SEQ ID NO: 46 - DNA, 54 bp, synthetic construct
  source 1..54: mol_type = other DNA
cagaatcagaattgacccttttctcgatatgtttttctgtgatttttttttttc

SEQ ID NO: 47 - DNA, 50 bp, synthetic construct
  source 1..50: mol_type = other DNA
gaaatggcgagtattgataatgagtgaatggtatgaacatggacatgagc

SEQ ID NO: 48 - DNA, 49 bp, synthetic construct
  source 1..49: mol_type = other DNA
catatcgagaaaagggtcaattctgattctgaatgtccattgtctcatg

SEQ ID NO: 49 - DNA, 48 bp, synthetic construct
  source 1..48: mol_type = other DNA
gttcataccattcactcattatcaatactcgccatttcaaagaatacg

SEQ ID NO: 50 - DNA, 46 bp, synthetic construct
  source 1..46: mol_type = other DNA
cagaatcagaattacttgattgttttgtccgattttcttgtttttc

SEQ ID NO: 51 - DNA, 41 bp, synthetic construct
  source 1..41: mol_type = other DNA
gagtattgataatgactagtaacgccgtatcgtgattaacg

SEQ ID NO: 52 - DNA, 45 bp, synthetic construct
  source 1..45: mol_type = other DNA
caaaacaatcaagtaattctgattctgaatgtccattgtctcatg

SEQ ID NO: 53 - DNA, 45 bp, synthetic construct
  source 1..45: mol_type = other DNA
cggcgttactagtcattatcaatactcgccatttcaaagaatacg

SEQ ID NO: 54 - DNA, 49 bp, synthetic construct
  source 1..49: mol_type = other DNA
cagaatcagaattcttgccttatgtggttttctattctattggatttac

SEQ ID NO: 55 - DNA, 48 bp, synthetic construct
  source 1..48: mol_type = other DNA
ctttgaaatggcgagtattgataatgagccgtcgaaaaggatctcgtc

SEQ ID NO: 56 - DNA, 45 bp, synthetic construct
  source 1..45: mol_type = other DNA
ccacataaggcaagaattctgattctgaatgtccattgtctcatg

SEQ ID NO: 57 - DNA, 46 bp, synthetic construct
  source 1..46: mol_type = other DNA
ccttttcgacggctcattatcaatactcgccatttcaaagaatacg

SEQ ID NO: 58 - DNA, 51 bp, synthetic construct
  source 1..51: mol_type = other DNA
cagcagccattttgttttgtgtgtaaatttagtgaagtactgttttttgtg

SEQ ID NO: 59 - DNA, 41 bp, synthetic construct
  source 1..41: mol_type = other DNA
gaaatggcgagtattgataatgagaaaccacaccgtggggc

SEQ ID NO: 60 - DNA, 39 bp, synthetic construct
  source 1..39: mol_type = other DNA
cacaaaacaaaatggctgctggttctattactactttgc

SEQ ID NO: 61 - DNA, 43 bp, synthetic construct
  source 1..43: mol_type = other DNA
gtgtggtttctcattatcaatactcgccatttcaaagaatacg

SEQ ID NO: 62 - DNA, 59 bp, synthetic construct
  source 1..59: mol_type = other DNA
cagcagccattgttttatatttgttgtaaaaagtagataattacttccttgatgatctg

SEQ ID NO: 63 - DNA, 52 bp, synthetic construct
  source 1..52: mol_type = other DNA
gaaatggcgagtattgataatgagtgagtaaggaaagagtgaggaactatcg

SEQ ID NO: 64 - DNA, 41 bp, synthetic construct
  source 1..41: mol_type = other DNA
caaatataaaacaatggctgctggttctattactactttgc

SEQ ID NO: 65 - DNA, 43 bp, synthetic construct; identical to SEQ ID NO: 25
  source 1..43: mol_type = other DNA

SEQ ID NO: 66 - DNA, 50 bp, synthetic construct
  source 1..50: mol_type = other DNA
cagcagccattttgtttgtttatgtgtgtttattcgaaactaagttcttg

SEQ ID NO: 67 - DNA, 46 bp, synthetic construct
  source 1..46: mol_type = other DNA
gttctagaatccgataaaaaacacgctttttcagttcgagtttatc

SEQ ID NO: 68 - DNA, 42 bp, synthetic construct
  source 1..42: mol_type = other DNA
cataaacaaacaaaatggctgctggttctattactactttgc

SEQ ID NO: 69 - DNA, 38 bp, synthetic construct
  source 1..38: mol_type = other DNA
gcgtgttttttatcggattctagaactagtggatcccc

SEQ ID NO: 70 - DNA, 58 bp, synthetic construct
  source 1..58: mol_type = other DNA
cagcagccattttgaatatgtattacttggttatggttatatatgacaaaagaaaaag

SEQ ID NO: 71 - DNA, 51 bp, synthetic construct; identical to SEQ ID NO: 27
  source 1..51: mol_type = other DNA

SEQ ID NO: 72 - DNA, 38 bp, synthetic construct
  source 1..38: mol_type = other DNA
catattcaaaatggctgctggttctattactactttgc

SEQ ID NO: 73 - DNA, 43 bp, synthetic construct
  source 1..43: mol_type = other DNA
gtattgttattcattatcaatactcgccatttcaaagaatacg

SEQ ID NO: 74 - DNA, 53 bp, synthetic construct
  source 1..53: mol_type = other DNA
ccagcagccatttttgagggaatattcaactgtttttttttatcatgttgatg

SEQ ID NO: 75 - DNA, 47 bp, synthetic construct
  source 1..47: mol_type = other DNA
gagtattgataatgatttgccagcttactatccttcttgaaaatatg

SEQ ID NO: 76 - DNA, 44 bp, synthetic construct
  source 1..44: mol_type = other DNA
gaatattccctcaaaaatggctgctggttctattactactttgc

SEQ ID NO: 77 - DNA, 46 bp, synthetic construct; identical to SEQ ID NO: 37
  source 1..46: mol_type = other DNA

SEQ ID NO: 78 - DNA, 43 bp, synthetic construct
  source 1..43: mol_type = other DNA
cagcagccattttttctattctgctgtttgctgtacctctttc

SEQ ID NO: 79 - DNA, 37 bp, synthetic construct
  source 1..37: mol_type = other DNA
gtattgataatgagtcattgtaccgcgaactttggac

SEQ ID NO: 80 - DNA, 39 bp, synthetic construct
  source 1..39: mol_type = other DNA
gaatagaaaaaatggctgctggttctattactactttgc

SEQ ID NO: 81 - DNA, 43 bp, synthetic construct
  source 1..43: mol_type = other DNA
gtacaatgactcattatcaatactcgccatttcaaagaatacg

SEQ ID NO: 82 - DNA, 55 bp, synthetic construct
  source 1..55: mol_type = other DNA
ccagcagccatgtttagttaattatagttcgttgaccgtatattctaaaaacaag

SEQ ID NO: 83 - DNA, 45 bp, synthetic construct
  source 1..45: mol_type = other DNA
gagtattgataatgaggggccgtatacttacatatagtagatgtc

SEQ ID NO: 84 - DNA, 44 bp, synthetic construct
  source 1..44: mol_type = other DNA
ctataattaactaaacatggctgctggttctattactactttgc

SEQ ID NO: 85 - DNA, 45 bp, synthetic construct; identical to SEQ ID NO: 45
  source 1..45: mol_type = other DNA

SEQ ID NO: 86 - DNA, 51 bp, synthetic construct
  source 1..51: mol_type = other DNA
cagcagccatgacccttttctcgatatgtttttctgtgatttttttttttc

SEQ ID NO: 87 - DNA, 44 bp, synthetic construct
  source 1..44: mol_type = other DNA
gcgagtattgataatgagtgaatggtatgaacatggacatgagc

SEQ ID NO: 88 - DNA, 38 bp, synthetic construct
  source 1..38: mol_type = other DNA
gaaaagggtcatggctgctggttctattactactttgc

SEQ ID NO: 89 - DNA, 45 bp, synthetic construct
  source 1..45: mol_type = other DNA
cataccattcactcattatcaatactcgccatttcaaagaatacg

SEQ ID NO: 90 - DNA, 47 bp, synthetic construct
  source 1..47: mol_type = other DNA
ccagcagccatacttgattgttttgtccgattttcttgtttttcttg

SEQ ID NO: 91 - DNA, 45 bp, synthetic construct
  source 1..45: mol_type = other DNA
gtattgataatgactagtaacgccgtatcgtgattaacgtattac

SEQ ID NO: 92 - DNA, 42 bp, synthetic construct
  source 1..42: mol_type = other DNA
caaaacaatcaagtatggctgctggttctattactactttgc

SEQ ID NO: 93 - DNA, 44 bp, synthetic construct
  source 1..44: mol_type = other DNA
ggcgttactagtcattatcaatactcgccatttcaaagaatacg

SEQ ID NO: 94 - DNA, 49 bp, synthetic construct
  source 1..49: mol_type = other DNA
ccagcagccatcttgccttatgtggttttctattctattggatttactc

SEQ ID NO: 95 - DNA, 34 bp, synthetic construct
  source 1..34: mol_type = other DNA
gtattgataatgagccgtcgaaaaggatctcgtc

SEQ ID NO: 96 - DNA, 39 bp, synthetic construct
  source 1..39: mol_type = other DNA
cataaggcaagatggctgctggttctattactactttgc

SEQ ID NO: 97 - DNA, 45 bp, synthetic construct
  source 1..45: mol_type = other DNA
cttttcgacggctcattatcaatactcgccatttcaaagaatacg

SEQ ID NO: 98 - DNA, 50 bp, synthetic construct
  source 1..50: mol_type = other DNA
gaacccaaagatttgttttgtgtgtaaatttagtgaagtactgttttttg

SEQ ID NO: 99 - DNA, 36 bp, synthetic construct
  source 1..36: mol_type = other DNA
ggcgagtattgataatgagaaaccacaccgtggggc

SEQ ID NO: 100 - DNA, 42 bp, synthetic construct
  source 1..42: mol_type = other DNA
cacacaaaacaaatctttgggttctttgactattgctgaacc

SEQ ID NO: 101 - DNA, 45 bp, synthetic construct; identical to SEQ ID NO: 21
  source 1..45: mol_type = other DNA

SEQ ID NO: 102 - DNA, 60 bp, synthetic construct
  source 1..60: mol_type = other DNA
gaacccaaagatgttttatatttgttgtaaaaagtagataattacttccttgatgatctg

SEQ ID NO: 103 - DNA, 56 bp, synthetic construct
  source 1..56: mol_type = other DNA
ctttgaaatggcgagtattgataatgagtgagtaaggaaagagtgaggaactatcg

SEQ ID NO: 104 - DNA, 42 bp, synthetic construct
  source 1..42: mol_type = other DNA
caaatataaaacatctttgggttctttgactattgctgaacc

SEQ ID NO: 105 - DNA, 43 bp, synthetic construct; identical to SEQ ID NO: 25
  source 1..43: mol_type = other DNA

SEQ ID NO: 106 - DNA, 55 bp, synthetic construct
  source 1..55: mol_type = other DNA
caaagaacccaaagatttgtttgtttatgtgtgtttattcgaaactaagttcttg

SEQ ID NO: 107 - DNA, 55 bp, synthetic construct
  source 1..55: mol_type = other DNA
ctagttctagaatccgataaaaaacacgctttttcagttcgagtttatcattatc

SEQ ID NO: 108 - DNA, 47 bp, synthetic construct
  source 1..47: mol_type = other DNA
cacacataaacaaacaaatctttgggttctttgactattgctgaacc

SEQ ID NO: 109 - DNA, 46 bp, synthetic construct
  source 1..46: mol_type = other DNA
ctgaaaaagcgtgttttttatcggattctagaactagtggatcccc

SEQ ID NO: 110 - DNA, 59 bp, synthetic construct
  source 1..59: mol_type = other DNA
gaacccaaagatttgaatatgtattacttggttatggttatatatgacaaaagaaaaag

SEQ ID NO: 111 - DNA, 56 bp, synthetic construct
  source 1..56: mol_type = other DNA
ggcgagtattgataatgaataacaatactgacagtactaaataattgcctacttgg

SEQ ID NO: 112 - DNA, 45 bp, synthetic construct
  source 1..45: mol_type = other DNA
gtaatacatattcaaatctttgggttctttgactattgctgaacc

SEQ ID NO: 113 - DNA, 45 bp, synthetic construct
  source 1..45: mol_type = other DNA
cagtattgttattcattatcaatactcgccatttcaaagaatacg

SEQ ID NO: 114 - DNA, 54 bp, synthetic construct
  source 1..54: mol_type = other DNA
caaagaacccaaagattttgagggaatattcaactgtttttttttatcatgttg

SEQ ID NO: 115 - DNA, 50 bp, synthetic construct
  source 1..50: mol_type = other DNA
ggcgagtattgataatgatttgccagcttactatccttcttgaaaatatg

SEQ ID NO: 116 - DNA, 50 bp, synthetic construct
  source 1..50: mol_type = other DNA
cagttgaatattccctcaaaatctttgggttctttgactattgctgaacc

SEQ ID NO: 117 - DNA, 50 bp, synthetic construct
  source 1..50: mol_type = other DNA
gatagtaagctggcaaatcattatcaatactcgccatttcaaagaatacg

SEQ ID NO: 118 - DNA, 46 bp, synthetic construct
  source 1..46: mol_type = other DNA
gtcaaagaacccaaagatttttctattctgctgtttgctgtacctc

SEQ ID NO: 119 - DNA, 42 bp, synthetic construct; identical to SEQ ID NO: 39
  source 1..42: mol_type = other DNA

SEQ ID NO: 120 - DNA, 45 bp, synthetic construct
  source 1..45: mol_type = other DNA
cagcagaatagaaaaatctttgggttctttgactattgctgaacc

SEQ ID NO: 121 - DNA, 45 bp, synthetic construct
  source 1..45: mol_type = other DNA
cggtacaatgactcattatcaatactcgccatttcaaagaatacg

SEQ ID NO: 122 - DNA, 55 bp, synthetic construct
  source 1..55: mol_type = other DNA
gaacccaaagagtttagttaattatagttcgttgaccgtatattctaaaaacaag

SEQ ID NO: 123 - DNA, 49 bp, synthetic construct
  source 1..49: mol_type = other DNA
cgagtattgataatgaggggccgtatacttacatatagtagatgtcaag

SEQ ID NO: 124 - DNA, 45 bp, synthetic construct
  source 1..45: mol_type = other DNA
ctataattaactaaactctttgggttctttgactattgctgaacc

SEQ ID NO: 125 - DNA, 45 bp, synthetic construct; identical to SEQ ID NO: 45
  source 1..45: mol_type = other DNA

SEQ ID NO: 126 - DNA, 56 bp, synthetic construct
  source 1..56: mol_type = other DNA
caaagaacccaaagagacccttttctcgatatgtttttctgtgatttttttttttc

SEQ ID NO: 127 - DNA, 45 bp, synthetic construct
  source 1..45: mol_type = other DNA
ggcgagtattgataatgagtgaatggtatgaacatggacatgagc

SEQ ID NO: 128 - DNA, 42 bp, synthetic construct
  source 1..42: mol_type = other DNA
cgagaaaagggtctctttgggttctttgactattgctgaacc

SEQ ID NO: 129 - DNA, 45 bp, synthetic construct; identical to SEQ ID NO: 89
  source 1..45: mol_type = other DNA

SEQ ID NO: 130 - DNA, 48 bp, synthetic construct
  source 1..48: mol_type = other DNA
caaagaacccaaagaacttgattgttttgtccgattttcttgtttttc

SEQ ID NO: 131 - DNA, 48 bp, synthetic construct
  source 1..48: mol_type = other DNA
cgagtattgataatgactagtaacgccgtatcgtgattaacgtattac

SEQ ID NO: 132 - DNA, 43 bp, synthetic construct
  source 1..43: mol_type = other DNA
caaaacaatcaagttctttgggttctttgactattgctgaacc

SEQ ID NO: 133 - DNA, 43 bp, synthetic construct
  source 1..43: mol_type = other DNA
gcgttactagtcattatcaatactcgccatttcaaagaatacg

SEQ ID NO: 134 - DNA, 51 bp, synthetic construct
  source 1..51: mol_type = other DNA
caaagaacccaaagacttgccttatgtggttttctattctattggatttac

SEQ ID NO: 135 - DNA, 44 bp, synthetic construct
  source 1..44: mol_type = other DNA
gaaatggcgagtattgataatgagccgtcgaaaaggatctcgtc

SEQ ID NO: 136 - DNA, 43 bp, synthetic construct
  source 1..43: mol_type = other DNA
ccacataaggcaagtctttgggttctttgactattgctgaacc

SEQ ID NO: 137 - DNA, 45 bp, synthetic construct; identical to SEQ ID NO: 97
  source 1..45: mol_type = other DNA

SEQ ID NO: 138 - DNA, 50 bp, synthetic construct
  source 1..50: mol_type = other DNA
gcaggaaacattttgttttgtgtgtaaatttagtgaagtactgttttttg

SEQ ID NO: 139 - DNA, 45 bp, synthetic construct; identical to SEQ ID NO: 19
  source 1..45: mol_type = other DNA

SEQ ID NO: 140 - DNA, 39 bp, synthetic construct
  source 1..39: mol_type = other DNA
cacacaaaacaaaatgtttcctgctatgccattgtcttc

SEQ ID NO: 141 - DNA, 45 bp, synthetic construct; identical to SEQ ID NO: 21
  source 1..45: mol_type = other DNA

SEQ ID NO: 142 - DNA, 60 bp, synthetic construct
  source 1..60: mol_type = other DNA
gcaggaaacattgttttatatttgttgtaaaaagtagataattacttccttgatgatctg

SEQ ID NO: 143 - DNA, 52 bp, synthetic construct; identical to SEQ ID NO: 63
  source 1..52: mol_type = other DNA

SEQ ID NO: 144 - DNA, 49 bp, synthetic construct
  source 1..49: mol_type = other DNA
ctttttacaacaaatataaaacaatgtttcctgctatgccattgtcttc

SEQ ID NO: 145 - DNA, 47 bp, synthetic construct
  source 1..47: mol_type = other DNA
ctttccttactcactcattatcaatactcgccatttcaaagaatacg

SEQ ID NO: 146 - DNA, 51 bp, synthetic construct
  source 1..51: mol_type = other DNA
gcaggaaacattttgtttgtttatgtgtgtttattcgaaactaagttcttg

SEQ ID NO: 147 - DNA, 51 bp, synthetic construct
  source 1..51: mol_type = other DNA
cactagttctagaatccgataaaaaacacgctttttcagttcgagtttatc

SEQ ID NO: 148 - DNA, 40 bp, synthetic construct
  source 1..40: mol_type = other DNA
cataaacaaacaaaatgtttcctgctatgccattgtcttc

SEQ ID NO: 149 - DNA, 38 bp, synthetic construct; identical to SEQ ID NO: 69
  source 1..38: mol_type = other DNA

SEQ ID NO: 150 - DNA, 59 bp, synthetic construct
  source 1..59: mol_type = other DNA
gcaggaaacattttgaatatgtattacttggttatggttatatatgacaaaagaaaaag

SEQ ID NO: 151 - DNA, 56 bp, synthetic construct; identical to SEQ ID NO: 111
  source 1..56: mol_type = other DNA

SEQ ID NO: 152 - DNA, 45 bp, synthetic construct
  source 1..45: mol_type = other DNA
caagtaatacatattcaaaatgtttcctgctatgccattgtcttc

SEQ ID NO: 153 - DNA, 45 bp, synthetic construct; identical to SEQ ID NO: 113
  source 1..45: mol_type = other DNA

SEQ ID NO: 154 - DNA, 57 bp, synthetic construct
  source 1..57: mol_type = other DNA
catagcaggaaacatttttgagggaatattcaactgtttttttttatcatgttgatg

SEQ ID NO: 155 - DNA, 50 bp, synthetic construct; identical to SEQ ID NO: 115
  source 1..50: mol_type = other DNA

SEQ ID NO: 156 - DNA, 42 bp, synthetic construct
  source 1..42: mol_type = other DNA
gaatattccctcaaaaatgtttcctgctatgccattgtcttc

SEQ ID NO: 157 - DNA, 46 bp, synthetic construct; identical to SEQ ID NO: 37
  source 1..46: mol_type = other DNA

SEQ ID NO: 158 - DNA, 44 bp, synthetic construct
  source 1..44: mol_type = other DNA
catagcaggaaacattttttctattctgctgtttgctgtacctc

SEQ ID NO: 159 - DNA, 42 bp, synthetic construct; identical to SEQ ID NO: 39
  source 1..42: mol_type = other DNA

SEQ ID NO: 160 - DNA, 40 bp, synthetic construct
  source 1..40: mol_type = other DNA
gcagaatagaaaaaatgtttcctgctatgccattgtcttc

SEQ ID NO: 161 - DNA, 45 bp, synthetic construct; identical to SEQ ID NO: 121
  source 1..45: mol_type = other DNA

SEQ ID NO: 162 - DNA, 60 bp, synthetic construct
  source 1..60: mol_type = other DNA
gcatagcaggaaacatgtttagttaattatagttcgttgaccgtatattctaaaaacaag

SEQ ID NO: 163 - DNA, 53 bp, synthetic construct; identical to SEQ ID NO: 43
  source 1..53: mol_type = other DNA

SEQ ID NO: 164 - DNA, 42 bp, synthetic construct
  source 1..42: mol_type = other DNA
ctataattaactaaacatgtttcctgctatgccattgtcttc

SEQ ID NO: 165 - DNA, 45 bp, synthetic construct; identical to SEQ ID NO: 45
  source 1..45: mol_type = other DNA

SEQ ID NO: 166 - DNA, 56 bp, synthetic construct
  source 1..56: mol_type = other DNA
catagcaggaaacatgacccttttctcgatatgtttttctgtgatttttttttttc

SEQ ID NO: 167 - DNA, 50 bp, synthetic construct; identical to SEQ ID NO: 47
  source 1..50: mol_type = other DNA

SEQ ID NO: 168 - DNA, 50 bp, synthetic construct
  source 1..50: mol_type = other DNA
gaaaaacatatcgagaaaagggtcatgtttcctgctatgccattgtcttc

SEQ ID NO: 169 - DNA, 59 bp, synthetic construct
  source 1..59: mol_type = other DNA
cataccattcactcattatcaatactcgccatttcaaagaatacgtaaataattaatag

SEQ ID NO: 170 - DNA, 48 bp, synthetic construct
  source 1..48: mol_type = other DNA
catagcaggaaacatacttgattgttttgtccgattttcttgtttttc

SEQ ID NO: 171 - DNA, 44 bp, synthetic construct
  source 1..44: mol_type = other DNA
ggcgagtattgataatgactagtaacgccgtatcgtgattaacg

SEQ ID NO: 172 - DNA, 53 bp, synthetic construct
  source 1..53: mol_type = other DNA
caagaaaatcggacaaaacaatcaagtatgtttcctgctatgccattgtcttc

SEQ ID NO: 173 - DNA, 59 bp, synthetic construct
  source 1..59: mol_type = other DNA
cggcgttactagtcattatcaatactcgccatttcaaagaatacgtaaataattaatag

SEQ ID NO: 174 - DNA, 51 bp, synthetic construct
  source 1..51: mol_type = other DNA
catagcaggaaacatcttgccttatgtggttttctattctattggatttac

SEQ ID NO: 175 - DNA, 44 bp, synthetic construct; identical to SEQ ID NO: 135
  source 1..44: mol_type = other DNA

SEQ ID NO: 176 - DNA, 45 bp, synthetic construct
  source 1..45: mol_type = other DNA
gaaaaccacataaggcaagatgtttcctgctatgccattgtcttc

SEQ ID NO: 177 - DNA, 46 bp, synthetic construct; identical to SEQ ID NO: 57
  source 1..46: mol_type = other DNA

SEQ ID NO: 178 - DNA, 25 bp, synthetic construct
  source 1..25: mol_type = other DNA
gggtcttctaatgctgaaagttcac

SEQ ID NO: 179 - DNA, 60 bp, synthetic construct
  source 1..60: mol_type = other DNA
atccgctcacaattccacaatatataatttaatcctgtaaatatactgcttcaaagtttc

SEQ ID NO: 180 - DNA, 59 bp, synthetic construct
  source 1..59: mol_type = other DNA
agcagtatatttacaggattaaattatatattgtggaattgtgagcggataacaatttc

SEQ ID NO: 181 - DNA, 54 bp, synthetic construct
  source 1..54: mol_type = other DNA
aagttactataaaatatactgcggtatatatgcgtaaggagaaaataccgcatc

SEQ ID NO: 182 - DNA, 60 bp, synthetic construct
  source 1..60: mol_type = other DNA
cggtattttctccttacgcatatataccgcagtatattttatagtaactttattcttttg

SEQ ID NO: 183 - DNA, 60 bp, synthetic construct; identical to SEQ ID NO: 182
  source 1..60: mol_type = other DNA

SEQ ID NO: 184 - DNA, 20 bp, synthetic construct
  source 1..20: mol_type = other DNA
gaagggatcggatttggctg

SEQ ID NO: 185 - DNA, 50 bp, synthetic construct
  source 1..50: mol_type = other DNA
gtaatcatggtcatagctgtttcctggctaaacttttcttacttctccgc

SEQ ID NO: 186 - DNA, 52 bp, synthetic construct
  source 1..52: mol_type = other DNA
gcggagaagtaagaaaagtttagccaggaaacagctatgaccatgattacgc

SEQ ID NO: 187 - DNA, 51 bp, synthetic construct
  source 1..51: mol_type = other DNA
caaataaaattcaaacaaaaaccaaaactaactaaaacgacggccagtgcc

SEQ ID NO: 188 - DNA, 52 bp, synthetic construct
  source 1..52: mol_type = other DNA
ggcactggccgtcgttttagttagttttggtttttgtttgaattttatttgg

SEQ ID NO: 189 - DNA, 36 bp, synthetic construct
  source 1..36: mol_type = other DNA
gttatgcttaatgaatttttgaatcagaaaaagaag

SEQ ID NO: 190 - DNA, 21 bp, synthetic construct
  source 1..21: mol_type = other DNA
ggaacagtgtgacatccaacc

SEQ ID NO: 191 - DNA, 59 bp, synthetic construct
  source 1..59: mol_type = other DNA
catggtcatagctgtttcctgttcttcatttagaaaaatttcagctgcttttttttttc

SEQ ID NO: 192 - DNA, 56 bp, synthetic construct
  source 1..56: mol_type = other DNA
aagcagctgaaatttttctaaatgaagaacaggaaacagctatgaccatgattacg

SEQ ID NO: 193 - DNA, 58 bp, synthetic construct
  source 1..58: mol_type = other DNA
taaaaaaacaaactaaacctaattctaacaagcaaagtaaaacgacggccagtgccaa

SEQ ID NO: 194 - DNA, 58 bp, synthetic construct
  source 1..58: mol_type = other DNA
cactggccgtcgttttactttgcttgttagaattaggtttagtttgtttttttattgg

SEQ ID NO: 195 - DNA, 20 bp, synthetic construct
  source 1..20: mol_type = other DNA
caactgccggctgaaagagc

SEQ ID NO: 196 - DNA, 31 bp, synthetic construct
  source 1..31: mol_type = other DNA
catggcctaagtattgtgaagtaatcaaatc

SEQ ID NO: 197 - DNA, 60 bp, synthetic construct
  source 1..60: mol_type = other DNA
catggtcatagctgtttcctgatagactttgacaaaaataacttacaaacactaaataag

SEQ ID NO: 198 - DNA, 55 bp, synthetic construct
  source 1..55: mol_type = other DNA
gtttgtaagttatttttgtcaaagtctatcaggaaacagctatgaccatgattac

SEQ ID NO: 199 - DNA, 48 bp, synthetic construct
  source 1..48: mol_type = other DNA
ctaacgaagaataaggctgaagcaattgtttaaaacgacggccagtgc

SEQ ID NO: 200 - DNA, 50 bp, synthetic construct
  source 1..50: mol_type = other DNA
tggcactggccgtcgttttaaacaattgcttcagccttattcttcgttag

SEQ ID NO: 201 - DNA, 25 bp, synthetic construct
  source 1..25: mol_type = other DNA
cattccttctgcaataggcgcaatc

(19) United States
(12) Patent Application Publication
(10) Pub. No.: US 2025/0019735 A1
(43) Pub. Date: Jan. 16, 2025

(54) Saccharomyces Cerevisiae Lysate Containing One or More Cytokines, Preparation Method Therefor and Application Thereof in Cell-Cultured Meat

(71) Applicant: Jiangnan University, Wuxi (CN)

(72) Inventors: Xin Guan, Wuxi (CN); Jingwen Zhou, Wuxi (CN); Qingzi Lei, Wuxi (CN); Jian Chen, Wuxi (CN); Guocheng Du, Wuxi (CN)

(21) Appl. No.: 18/898,838

(22) Filed: Sep. 27, 2024

Related U.S. Application Data
(63) Continuation of application No. PCT/CN2023/113544, filed on Aug. 17, 2023.

(30) Foreign Application Priority Data
Jan. 10, 2023 (CN) 2023100310295

(51) Int. Cl. C12P 21/02; C07K 14/485; C07K 14/49; C07K 14/50; C07K 14/65

(52) U.S. Cl. CPC C12P 21/02; C07K 14/485; C07K 14/49; C07K 14/503; C07K 14/65

(57) ABSTRACT

The disclosure discloses a Saccharomyces cerevisiae lysate containing one or more cytokines, a preparation method therefor and an application thereof in cell-cultured meat, and belongs to the technical field of genetic engineering and cell-cultured meat. The disclosure provides a method for further increasing recombinant cytokine yields using the GRAS strain of S. cerevisiae recombinantly expressing a single cytokine or co-expressing a combination of cytokines through promoter optimization, knockout of endogenous protease in yeast, genome-integrated expression and other means. The yeast lysate may be directly used for muscle stem cell culture after the process such as filtration, sterilization, and cytokine concentration measurement, effectively promoting muscle stem cell proliferation. The disclosure avoids the complex purification process in the production of recombinant cytokines, reduces the cost of cytokine production, and provides new ideas for large-scale low-cost development of cell-cultured meat.

### REFERENCE TO SEQUENCE LISTING

The instant application contains a Sequence Listing in XML format as a file named “YGHY-2023-69-SEQ.xml”, created on Sep. 20, 2024, of 183,749 bytes in size, and which is hereby incorporated by reference in its entirety.

### TECHNICAL FIELD

The disclosure belongs to the technical field of genetic engineering and cell-cultured meat, and relates to a S. cerevisiae lysate containing one or more cytokines, a preparation method therefor and an application thereof in cell-cultured meat, and more specifically relates to the construction of a recombinant strain of S. cerevisiae that intracellularly expresses single or multiple cytokines, and a method of applying the strain to the production of cell-cultured meat immediately after being physically fragmented and sterilized.

### BACKGROUND

As the world's population continues to grow and the level of economic and social development of mankind continues to rise, the consumption of meat products will grow rapidly, and the traditional way of animal husbandry is facing enormous supply pressure. As an important component of future food products, a cell-cultured meat is a new protein resource and meat food that provides an efficient, safe and sustainable solution for animal protein supply, and is regarded as one of the most important means to address the future gap in the meat products. It is based on cell biology and tissue engineering, and is made by culturing animal cells in vitro to obtain muscle fibers, fats, and other parts that make up muscle tissue, and then collected and processed for food. It greatly improves the production efficiency of meat products, reduces energy consumption and minimizes greenhouse gas emissions, among other issues. The key technology lies in obtaining a large number of seed cells with differentiation potential, such as muscle stem cells and mesenchymal stem cells, at high efficiency and low cost in vitro. However, since the weak proliferation capacity of seed cells during the current culture in vitro cannot meet the amount of seed cells required by the cell-cultured meat industry, there is an urgent need to find an efficient and low-cost way to promote the rapid proliferation of seed cells in vitro.

Cytokines are small molecule polypeptides and glycoproteins synthesized and secreted by cells themselves, which have various physiological functions such as the regulation of cell growth, differentiation and maturation, and function maintenance. They have been widely used in the fields such as pharmaceuticals, tissue engineering, and future foods by regulating different pathways and thus regulating the process of cell proliferation and differentiation. When culturing various types of cells in vitro, the addition of suitable cytokines can promote cell growth and proliferation, maintain cell function, and reduce the concentration of serum as used. At the same time, there is a synergistic effect between different growth factors, and the synergistic effect of multiple growth factors can lead to better results in cell culture in vitro. It has been shown that during the culture of stem cells, the addition of single cytokine such as basic fibroblast growth factor (bFGF), epidermal growth factor (EGF), platelet-derived growth factor-AA (PDGF-AA), platelet-derived growth factor-BB (PDGF-BB), insulin-like growth factor 1 (IGF-1), long chain insulin-like growth factor (LR³-IGF-1), vascular endothelial growth factor (VEGF), hepatocyte growth factor (HGF), oncostatin M (OSM), interleukin-6 (IL-6) and transforming growth factor-β family (TGF-β family), or a combination thereof such as (EGF, PDGF-BB, LR³-IGF-1 and bFGF) and (IL-6, bFGF, IGF-1, PDGF-BB, VEGF and HGF) can effectively improve the proliferation efficiency of muscle stem cells in vitro and can effectively reduce the dependence of muscle stem cells on fetal bovine serum. However, the current commercially available cytokines are very expensive, accounting for 60% to 80% of the total cost of the medium, severely limiting their application to the low-cost production of the cell-cultured meat.

Currently, cytokines have been widely used in basic science research and biopharmaceuticals, and are mainly produced by recombinant expression using Escherichia coli and Pichia pastoris as hosts according to genetic engineering strategies. The main production processes are as follows: a. obtaining gene fragments of cytokines and constructing recombinant expression strains; b. fermenting the recombinant strains on a large scale and inducing the expression of cytokines; c. enriching and purifying the cytokines; and d. refining the cytokines, removing the endotoxins and freeze-drying to produce commercially available finished products. However, the application of the above cytokine production process in the field of cell-cultured meat faces 2 key challenges: 1) neither E. coli nor P. pastoris is a food-safe strain, and the application of recombinant cytokines expressed by them in cell-cultured meat production may pose additional food safety risks and uncertainties; and 2) as the scientific research and pharmaceutical industries have very strict purity standards and production specifications for cytokines, the fermentation broth of engineered bacteria needs to undergo a series of workup steps such as enrichment, purification and refinement to obtain highly pure cytokines, which brings about high purification costs and leads to high prices of recombinant cytokines. Therefore, there is an urgent need to develop a new strategy for cytokine production in the field of cell-cultured meat to simplify the production process and reduce the production cost as much as possible under the premise of guaranteeing the safety and effectiveness of the product, which is the key to the low-cost production of cell-cultured meat.

S. cerevisiae, as a Generally Recognized as Safe (GRAS) microorganism, possesses the ability to post-translationally process and modify proteins in eukaryotic cells, can express biologically active exogenous proteins in an even better fashion, grows rapidly, has a low cost of culture, is suitable for large-scale fermentation culture on an industrial scale, and has been widely used in the food and pharmaceutical industries with the potential for large-scale production of cytokines. Therefore, intracellular expression of one and more cytokines using S. cerevisiae as chassis cells, followed by a simple and low-cost workup procedure of physical fragmentation and filtration, and direct addition to the medium of seed cells of cultured meat will effectively promote cell proliferation in vitro and significantly reduce the cost of cytokine application, thus promoting the development of the cell-cultured meat industry.

### SUMMARY

To solve the problems such as weak proliferative capacity and high culture cost of seed cells of cell-cultured meat during the culture in vitro, complicated workup of commercially available cytokines promoting cell proliferation, high purification cost, and food safety hazard, the disclosure provides a method of applying S. cerevisiae that intracellularly express cytokines to the production of cell-cultured meat immediately after being physically fragmented. A yeast lysate containing cytokines is prepared by mainly using the GRAS strain of S. cerevisiae as the host for intracellular expression of single or multiple cytokines, and physically fragmenting the recombinant strain after fermentation via an instrument such as FastPrep-24™ or a high-pressure homogenizer, while maximizing the retention of cytokine activity. Subsequently, the yeast lysate is directly used for muscle stem cell culture after the process such as filtration, sterilization, and cytokine concentration measurement, promoting muscle stem cell proliferation in vitro while significantly reducing the cost of the medium (FIG. 1). Meanwhile, the disclosure provides a method for dramatically increasing cytokine yields by S. cerevisiae through promoter optimization, knockout of endogenous protease in yeast, genome-integrated expression and other means. The disclosure avoids the complex purification process in the production of recombinant cytokines, greatly reduces the cost of cytokine production, effectively promotes the in vitro proliferation of muscle stem cells, and contributes to the development of the cell-cultured meat industry. The specific technical solution is as follows:

A first object according to the disclosure is to provide a recombinant S. cerevisiae, where S. cerevisiae is used as a starting strain, the endogenous genes PEP4, YAP3, PRB1 and CYM1 are knocked out, and a cytokine expression cassette is overexpressed.

In one embodiment according to the disclosure, the cytokine expression cassette includes cytokine-encoding genes that are combined sequentially or concurrently and whose expression is initiated by a promoter.

In one embodiment according to the disclosure, the cytokine-encoding genes include a gene encoding basic fibroblast growth factor (bFGF), a gene encoding epidermal growth factor (EGF), a gene encoding platelet-derived growth factor-AA (PDGF-AA), a gene encoding platelet-derived growth factor-BB (PDGF-BB), a gene encoding insulin-like growth factor 1 (IGF-1), a gene encoding long chain insulin-like growth factor (LR³-IGF-1), a gene encoding vascular endothelial growth factor (VEGF), a gene encoding hepatocyte growth factor (HGF), a gene encoding oncostatin M (OSM), a gene encoding interleukin-6 (IL-6), and a gene encoding transforming growth factor-β family (TGF-β family).

In one embodiment according to the disclosure, the cytokine-encoding genes are derived from Homo sapiens, Sus scrofa, Bos taurus, Mus musculus, and Rattus norvegicus.

In one embodiment according to the disclosure, the cytokine-encoding genes are genes encoding the cytokines EGF, PDGF-BB, LR³-IGF-1 and bFGF.

In one embodiment according to the disclosure, the starting strain is S. cerevisiae C800.

In one embodiment according to the disclosure, the expression of the gene encoding the cytokine EGF is initiated by the promoter P_{GAL7}, the expression of the gene encoding the cytokine PDGF-BB is initiated by the promoter P_{FBA1}, the expression of the gene encoding the cytokine LR³-IGF-1 is initiated by the promoter P_{PGK1}, and the expression of the gene encoding the cytokine bFGF is initiated by the promoter P_{ADE2}.

In one embodiment according to the disclosure, the overexpression includes free expression or integrated expression.

In one embodiment according to the disclosure, the cytokine expression cassette is integrated into the S. cerevisiae genome at the multi-copy loci Ty1 and Ty2.

In one embodiment according to the disclosure, the Gene ID of the endogenous gene PEP4 is 855949, the Gene ID of the endogenous gene YAP3 is 850811, the Gene ID of the endogenous gene PRB1 is 856649, and the Gene ID of the endogenous gene CYM1 is 852041.

In one embodiment according to the disclosure, the genes encoding the cytokines EGF, PDGF-BB, LR³-IGF-1 and bFGF are all derived from H. sapiens, the Gene ID of the gene encoding the cytokine EGF is 1950; the Gene ID of the gene encoding the cytokine PDGF-BB is 5155; the Gene ID of the gene encoding the cytokine bFGF is 2247; and the nucleotide sequence of the gene encoding the cytokine LR³-IGF-1 is set forth in SEQ ID NO: 1.

A second object according to the disclosure is to provide a method of constructing the recombinant S. cerevisiae using S. cerevisiae as a starting strain, knocking out the endogenous genes PEP4, YAP3, PRB1 and CYM1, and overexpressing a cytokine expression cassette.

In one embodiment according to the disclosure, the cytokine expression cassette includes cytokine-encoding genes that are combined sequentially or concurrently and whose expression is initiated by a promoter.

In one embodiment according to the disclosure, the cytokine-encoding genes include a gene encoding basic fibroblast growth factor (bFGF), a gene encoding epidermal growth factor (EGF), a gene encoding platelet-derived growth factor-AA (PDGF-AA), a gene encoding platelet-derived growth factor-BB (PDGF-BB), a gene encoding insulin-like growth factor 1 (IGF-1), a gene encoding long chain insulin-like growth factor (LR³-IGF-1), a gene encoding vascular endothelial growth factor (VEGF), a gene encoding hepatocyte growth factor (HGF), a gene encoding oncostatin M (OSM), a gene encoding interleukin-6 (IL-6), and a gene encoding transforming growth factor-β family (TGF-β family).

In one embodiment according to the disclosure, the cytokine-encoding genes are derived from H. sapiens, S. scrofa, B. taurus, M. musculus, and R. norvegicus.

In one embodiment according to the disclosure, the expression of the gene encoding the cytokine EGF is initiated by the promoter P_{GAL7}, the expression of the gene encoding the cytokine PDGF-BB is initiated by the promoter P_{FBA1}, the expression of the gene encoding the cytokine LR³-IGF-1 is initiated by the promoter P_{PGK1}, and the expression of the gene encoding the cytokine bFGF is initiated by the promoter P_{ADE2}.

In one embodiment according to the disclosure, the overexpression includes free expression or integrated expression.

In one embodiment according to the disclosure, the starting strain is S. cerevisiae C800.

In one embodiment according to the disclosure, the genes encoding the cytokines EGF, PDGF-BB, LR³-IGF-1 and bFGF are all derived from H. sapiens, the Gene ID of the gene encoding the cytokine EGF is 1950; the Gene ID of the gene encoding the cytokine PDGF-BB is 5155; the Gene ID of the gene encoding the cytokine bFGF is 2247; and the nucleotide sequence of the gene encoding the cytokine LR³-IGF-1 is set forth in SEQ ID NO: 1.

In one embodiment according to the disclosure, the construction method includes integrating the expression elements of the genes encoding the cytokines EGF, PDGF-BB, LR³-IGF-1, and bFGF into the S. cerevisiae genome at the multi-copy locus Ty1, respectively, and screening to obtain the recombinant S. cerevisiae strains CB1E6, CB1P1, CB113, and CB1B2, which highly express the cytokines EGF, PDGF-BB, LR³-IGF-1, and bFGF.

In one embodiment according to the disclosure, the cytokine expression cassette is integrated into the S. cerevisiae genome at the multi-copy locus Ty1/Ty2.

A third object according to the disclosure is to provide a method for preparing a cytokine including:
(1) subjecting the recombinant S. cerevisiae to a fermentation culture to obtain a bacterial broth; and
(2) physically fragmenting the bacterial broth in a low temperature environment, removing the bacterial fragments, removing the endotoxins, filtering and sterilizing to obtain the cytokine.

In one embodiment according to the disclosure, the cytokine as prepared using the method is added directly to a medium for cell-cultured meat production without purification.

In one embodiment according to the disclosure, the application includes inoculating recombinant S. cerevisiae in a liquid YPD medium for fermentation culture.

In one embodiment according to the disclosure, the fermentation culture is carried out in a 3 L fermenter with a rotational speed of 600 rpm, an aeration rate of 3.0 vvm, and a pH of 5.5, when the initial glucose is depleted, a fed-batch fermentation is carried out at a rate of 5 mL/h and terminated after 62 h, and the fermentation is terminated when the bacterium is no longer growing.

In one embodiment according to the disclosure, the composition of the fed-batch fermentation medium is 400 g/L glucose, 18 g/L KH₂PO₄, 10.24 g/L MgSO₄·7H₂O, 7 g/L K₂SO₄, 0.56 g/L Na₂SO₃, 1 g/L leucine, 1 g/L histidine, 1 g/L tryptophan, 20 mL/L trace metal element solution, 24 mL/L vitamin solution, and the balance being water.

In one embodiment according to the disclosure, in step (2), the bacterial broth is centrifuged, a wet bacterium is harvested, washed with PBS 2 times and then resuspended again by PBS, the batch fragmentation of the yeast wet bacterium is carried out by using a high-pressure homogenizer under the pressure of 1000-1400 bar for 4-6 cycles, and the temperature of the high-pressure homogenizer is set as 4° C. to avoid the denaturation of cytokines by high temperatures.

A fourth object according to the disclosure is to provide an application of the recombinant S. cerevisiae or the method in the cell-cultured meat production.

In one embodiment according to the disclosure, the application includes inoculating recombinant S. cerevisiae in a medium for fermentation culture, obtaining a lysate of the recombinant S. cerevisiae at the end of the fermentation, sterilizing the collected lysate without purification, diluting to a certain concentration, adding directly to a medium, and applying to the culture of a seed cell of the cell-cultured meat;

alternatively, sterilizing the cytokine as prepared by the method, diluting to a certain concentration and directly applying to the culture of a seed cell of the cell-cultured meat.

In one embodiment according to the disclosure, the seed cell of the cell-cultured meat is a muscle stem cell.

In one embodiment according to the disclosure, the culture of the seed cell of the cell-cultured meat includes diluting the concentration of the lysate of the recombinant S. cerevisiae co-expressing the four cytokines to 1 g/L, then adding to a DMEM medium containing 5% FBS, filtering, sterilizing, and then applying to the culture of the muscle stem cell.

In one embodiment, the sterilization includes a sterilization by filtration.

In one embodiment according to the disclosure, the culture of the muscle stem cell using a medium containing yeast lysate can effectively promote the rapid proliferation of muscle stem cells to the same or an even greater extent than the commercially available cytokines at equivalent concentrations without affecting the differentiation potential of muscle stem cells.

### Beneficial Effects According to the Disclosure

Slow cell proliferation, severe serum dependence, high price of commercially available cytokines in components of medium and the like are the key issues hindering the development of cultured meat industry. The disclosure provides a recombinant S. cerevisiae intracellularly expressing four cytokines and a method of applying the recombinant S. cerevisiae to the production of cell-cultured meat immediately after physical fragmentation only. The cytokine yields are further improved by mainly using the GRAS strain of S. cerevisiae for recombinant expression of a single cytokine or co-expression of a cytokine combination, a promoter optimization and an integrated expression of multi-copy loci on the genome. A process for knocking out endogenous proteases in S. cerevisiae (PEP4, CYM1, YAP3, and PRB1) is also provided, which reduces intracellular degradation of cytokines and increases the yield of heterologously expressed cytokines in yeast. Among them, recombinant S. cerevisiae CPK2B2, which concurrently expresses four cytokines, possesses the highest cytokine yield of 18.35 mg/L, in which the yields of bFGF, EGF, PDGF-BB, and LR³-IGF-1 reach 9.56 mg/L, 0.87 mg/L, 1.28 mg/L, and 6.64 mg/L, respectively.

Based on the recombinant S. cerevisiae that can prepare four cytokines at high concentrations provided by the disclosure, the recombinant S. cerevisiae is physically fragmented using an instrument such as FastPrep-24™ or a high-pressure homogenizer to maximize the retention of cytokine activity. It can be directly used for muscle stem cell culture after the process such as filtration, sterilization, and cytokine concentration measurement, which promotes the efficient proliferation of muscle stem cells, avoids the complicated and costly purification process and greatly reduces the cost of cell culture. The lysate prepared from the recombinant S. cerevisiae provided by the disclosure reduces the cost in cell-cultured meat production to USD 0.3 per liter or less. The disclosure provides new ideas for large-scale low-cost development of cell-cultured meat and promotes the development of the culture meat industry.

### BRIEF DESCRIPTION OF FIGURES

FIG. 1 is a diagram showing the main technical roadmap of the disclosure;

FIG. 2 is a diagram showing the amplification results of the four cytokine gene fragments;

FIG. 3 is a diagram showing the results of a Western blot assay for four cytokines expressed by plasmids;

FIG. 4 is a diagram showing the results of a concentration measurement for four cytokines expressed by plasmids;

FIG. 5 is a diagram showing the different promoters and vector fragments as obtained during promoter optimization of the four cytokine expression systems;

FIG. 6 is a diagram showing the results of yields for four cytokines under different promoters;

FIG. 7 is a diagram showing the cytokine concentrations of different strains integrating the bFGF gene at the multi-copy locus;

FIG. 8 is a diagram showing the cytokine concentrations of different strains integrating the EGF gene at the multi-copy locus;

FIG. 9 is a diagram showing the cytokine concentrations of different strains integrating the LR³-IGF-1 gene at the multi-copy locus;

FIG. 10 is a diagram showing the cytokine concentrations of different strains integrating the PDGF-BB gene at the multi-copy locus;

FIG. 11 is a schematic diagram showing the sequential and concurrent expression cassettes for co-expression of the four cytokines;

FIG. 12 is a diagram showing the concentration of each cytokine for strains integrating four cytokines at different multi-copy loci;

FIG. 13 is a diagram showing the results of a Western blot assay for a lysate of strain CPK2B2 co-expressing the four cytokines;

FIG. 14 is a diagram showing the growth curves of the CPK2B2 strain under different conditions in a 3 L fermenter;

FIG. 15 is a diagram showing the effect of fed-batch fermentation on cytokine production by the CPK2B2 strain;

FIG. 16 is a diagram showing the effect of the four cytokines at different concentrations in promoting the proliferation of muscle stem cells;

FIG. 17 is a diagram showing the concentration of four cytokines contained in the CPK2B2 lysates at different concentrations;

FIG. 18 is a diagram showing the changes in the number of cells under different culture conditions;

FIG. 19 is a diagram showing the changes in cell viability under different culture conditions;

FIG. 20 is a diagram showing the cell cycle of each group of cells under different culture conditions;

FIG. 21 is a diagram showing the EdU detection results of each group of cells under different culture conditions;

FIG. 22 is a diagram showing the differentiation of each group of cells under different culture conditions.

FIG. 23 is a diagram showing the knockout results of endogenous protease PEP4, CYM1, YAP3, and PRB1 in S. cerevisiae;

FIG. 24 is a diagram showing the growth curves of different protease knockout strains versus the original strain; and

FIG. 25 is a diagram showing the yield variation of bFGF in different protease-deficient strains.

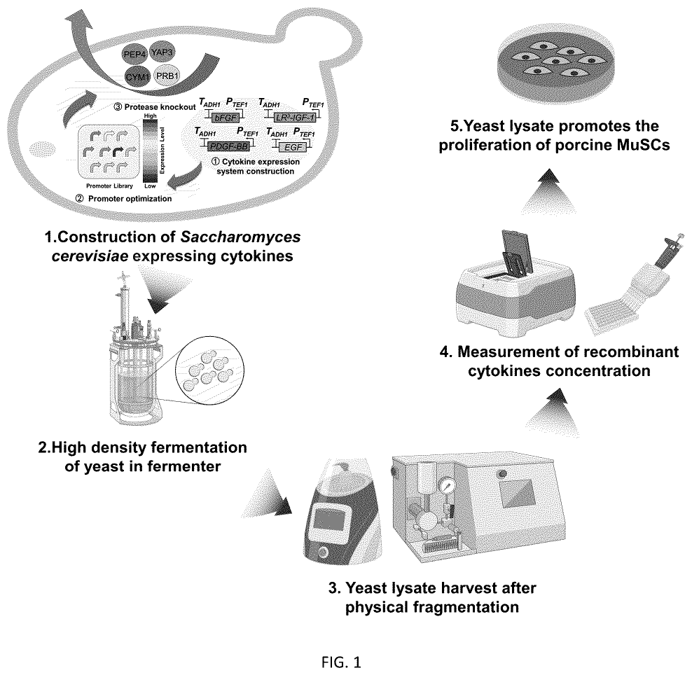

### DETAILED DESCRIPTION

The technical solutions described herein will be clearly and completely described below in connection with examples according to the disclosure, and it is clear that the described examples are only a part of the examples according to the disclosure and not all of them. Based on the examples according to the disclosure, all other examples obtained by a person of ordinary skill in the art without making creative labor fall within the scope of protection according to the disclosure.

S. cerevisiae C800 used in the following examples (MATa ura3-52, leu2-3,112, trp1-289, his3Δ1, Δgal80::kanMX)

The medium involved in the following examples are as follows:

LB liquid medium: peptone 10 g/L, yeast powder 5 g/L, NaCl 10 g/L, and the balance being water.

LB solid medium: peptone 10 g/L, yeast powder 5 g/L, NaCl 10 g/L, agar powder 20 g/L, and the balance being water.

SD liquid medium: Yeast Nutrition Base 6.74 g/L, glucose 20 g/L, amino acids (5 g/L uracil, 10 g/L tryptophan, 10 g/L leucine, and 10 g/L histidine, where corresponding amino acids can be deleted as needed), and the balance being water.

SD solid medium: Yeast Nutrition Base 6.74 g/L, glucose 20 g/L, amino acids (5 g/L uracil, 10 g/L tryptophan, 10 g/L leucine, and 10 g/L histidine, where corresponding amino acids can be deleted as needed), agar powder 20 g/L, and the balance being water.

YPD liquid medium: peptone 20 g/L, yeast powder 10 g/L, glucose 20 g/L, and the balance being water.

YPD solid medium: peptone 20 g/L, yeast powder 10 g/L, glucose 20 g/L, agar powder 20 g/L, and the balance being water.

Fed-batch fermentation medium: 400 g/L glucose, 18 g/L KH₂PO₄, 10.24 g/L MgSO₄·7H₂O, 7 g/L K₂SO₄, 0.56 g/L Na₂SO₃, 1 g/L leucine, 1 g/L histidine, 1 g/L tryptophan, 20 mL/L trace metal element solution, 24 mL/L vitamin solution, and the balance being water. The formulas of trace metal element solution and vitamin solution are described in the document (Gao S, Lyu Y, Zeng W, Du G, Zhou J, Chen J. Efficient Biosynthesis of (2S)-Naringenin from p-Coumaric Acid in Saccharomyces cerevisiae. J Agric Food Chem. 2020 Jan. 29; 68(4):1015-1021. doi: 10.1021/acs.jafc.9b05218. Epub 2020 Jan. 13. PMID: 31690080).

The microorganism culture methods involved in the following examples are as follows:

(1) E. coli: the culture was carried out in a LB liquid medium at 37° C. and 220 rpm.

(2) S. cerevisiae:

A. Shake flask culture: a single yeast colony was inoculated in a SD liquid medium, and cultured at 30° C. and 220 rpm for 16 h. The culture was inoculated with an inoculum size of 1% (v/v) into a 250 mL shake flask containing 25 mL of YPD medium and cultured at 30° C. and 220 rpm for 72 h.

B. Fermenter culture: after the yeast strain was activated by SD plate, a single clone was picked into a SD liquid medium, cultured at 30° C. and 220 rpm for 16 h, inoculated with an inoculum size of 1% (v/v) into a 500 mL shake flask containing 50 mL of YPD medium, sequentially cultured for 24 h, and then inoculated with an inoculum size of 2% (v/v) into a 3 L fermenter containing 1.5 L of YPD medium, and a scale-up culture was carried out under an aeration rate of 3.0 vvm at 600 rpm. The pH was maintained at 5.5 using 1 M HCL and aqueous ammonia. When the initial glucose was depleted, a fed-batch fermentation (fed-batch fermentation medium) was carried out at a rate of 5 mL/h and terminated after 62 h, and the fermentation was terminated when the bacterium was no longer growing.

The animal cell culture methods involved in the following examples are as follows:

Animal muscle stem cells were cultured in a DMEM medium containing 10% fetal bovine serum (FBS) at 37° C. under 5% CO₂. To examine the effect of a combination of recombinant cytokines, the yeast lysate was diluted to a concentration of 1-3 g/L, added to a DMEM medium containing 5% FBS, filtered and sterilized, and the muscle stem cells were cultured. To examine the differentiation ability of muscle stem cells, when the cell density reached about 90%, the above medium was replaced with a differentiation medium (DMEM medium containing 2% horse serum (HS)), and the muscle stem cell differentiation was observed after 4-5 d of culture.

### Western Blot Analysis:

S. cerevisiae lysate was denatured, separated by SDS-PAGE and then transferred to a PVDF membrane. The membrane was incubated with a blocking solution for 1 h and then incubated with a primary antibody at 4° C. overnight. The antibodies included anti-FGF2 (1:200, Santa Cruz Biotechnology), anti-EGF (1:200, Santa Cruz Biotechnology), anti-PDGF-B (1:200, Santa Cruz Biotechnology), and anti-IGF-1 (1:200, Santa Cruz Biotechnology). The membrane was then rinsed with TBST and incubated with HRP-conjugated Affinipure goat anti-mouse or anti-rabbit IgG (H+L) (1:2000, Proteintech Group) for 2 h at room temperature. The identified proteins were photographed and analyzed using a Tanon 5200 chemiluminescent imaging system (Tanon, Shanghai, China).

### Immunofluorescence Identification:

The muscle stem cells were inoculated in a confocal petri dish and cultured for 2 d. When an appropriate density was achieved, the cells were washed with a PBS buffer, added to a differentiation medium, and cultured for 3-4 d. After which, elongated multinucleated myotubes were observed under the microscope. The medium was aspirated and discarded. The culture was washed with PBS, fixed by adding 4% (w/v) paraformaldehyde (pre-cooled at 4° C.), and permeabilized at room temperature for 15 min. After which, the paraformaldehyde was removed and the culture was washed carefully with a PBS buffer, treated by adding 0.5% (v/v) Triton x-100 for 15 min, and washed carefully with a PBS buffer. A blocking solution (1% BSA and glycine with a final concentration of 22.52 mg/mL in PBST (PBS with 0.1% Tween20)) was added, and the culture was incubated for 30 min at room temperature and then wash carefully with a PBS buffer. MyHC antibodies (1:100) diluted in PBS with 1% bovine serum albumin were added separately and incubated at room temperature for 1 h followed by overnight at 4° C. After incubated overnight, the culture was left at room temperature for 1 h and then washed 3 times with PBS. A fluorescence-labeled secondary antibody diluted 1:200 in PBS solution with 1% BSA was added, and the culture was incubated at room temperature away from light for 1-1.5 h and then washed with PBS. 20 μM DAPI was added, and the culture was incubated at room temperature away from light for 10 min and then washed with PBS. An antifade mounting medium was added and the culture was photographed for observation under a fluorescence microscope.

### Cytokine Yield Assay:

The yield of recombinantly expressed cytokines was detected by corresponding ELISA kits including Human FGF2 ELISA Kit (KE00129, ProteinTech, USA), Human PDGF-BB ELISA Kit (KE00161, ProteinTech, USA), Human EGF ELISA Kit (KE00138, ProteinTech, USA) and Human LR³-IGF-1 ELISA kit (Jiangsu Meimian Industrial Co. Ltd., Yancheng, China).

### MTT Assay:

The incubation was carried out in the corresponding medium to be tested for 2 d, and then 20 μL of MTT solution (5 mg/mL) was added to each well. The incubation was continued for 4 h, and then the supernatant was discarded. 200 μL of DMSO was added to dissolve the formazan, and the absorbance value at 490 nm was detected using an enzyme-linked immunosorbent assay analyzer (BioTek, Winooski, VT, USA). Cell viability (%) was defined as the ratio of the absorbance value of cells in the treatment group to that of the control group.

### Example 1: Heterologous Expression of a Single Cytokine in S. cerevisiae

To construct a cytokine expression system in S. cerevisiae, 4 cytokine fragments were synthesized based on the genes encoding bFGF, EGF, PDGF-BB, and LR³-IGF-1 in the NCBI library, respectively. The genes encoding the cytokines EGF, PDGF-BB, LR³-IGF-1 and bFGF are all derived from H. sapiens, and the Gene ID of the gene encoding the cytokine EGF is 1950; the Gene ID of the gene encoding the cytokine PDGF-BB is 5155; the Gene ID of the gene encoding the cytokine bFGF is 2247; and the nucleotide sequence of the gene encoding the cytokine LR³-IGF-1 is set forth in SEQ ID NO: 1.

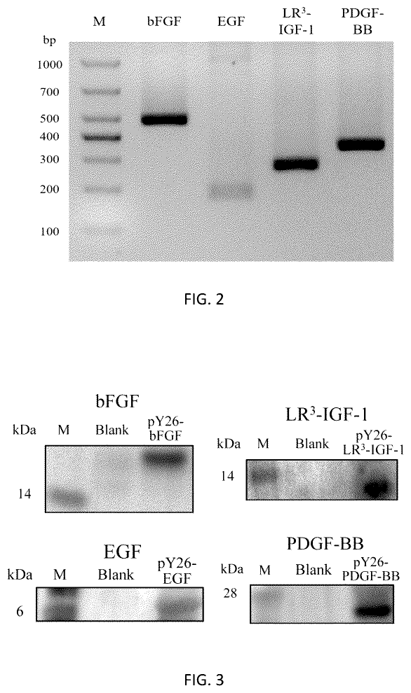

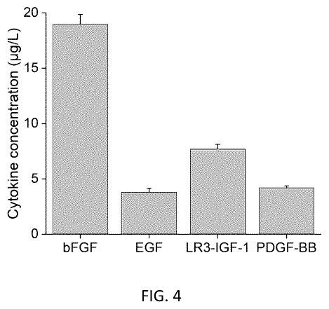

4 cytokine fragments were amplified from the synthesized gene fragments, respectively, and the results showed that the correctly sized bFGF, EGF, PDGF-BB, and LR3-IGF-1 fragments were successfully obtained (FIG. 2). The expression vector pY26 was used as a template to construct four cytokine expression plasmids, respectively. Homologous arms were designed at both ends of the insertion of the cytokine gene fragments and the corresponding pY26 vector fragments, respectively. 4 cytokine fragments were respectively integrated into the expression vector pY26 to obtain an expression plasmid of pY26-Cytokine, and the primers were shown in Table 1. The pY26-Cytokine plasmid as constructed was amplified, sequenced, and aligned correctly, and then introduced into S. cerevisiae C800 for expression. The bacterium was fermented in a YPD medium for 2 d, then collected, washed with PBS, and fragmented. After which, the lysate was collected for protein expression identification. The WB results of 4 cytokine-specific protein expression showed that there were specific bands at the positions of corresponding proteins. This indicated that all four cytokines had been successfully expressed, and the cytokine expression plasmid was successfully constructed (FIG. 3). The expression yields of bFGF, EGF, PDGF-BB, and LR³-IGF-1 plasmids were 18.9 μg/L, 3.8 μg/L, 7.7 μg/L, and 4.2 μg/L, respectively, as determined by the corresponding Elisa kit (FIG. 4).

TABLE 1
Primers for cytokine integration in plasmids

| Name | SEQ ID NO: | Sequence (5′-3′) |
|---|---|---|
| EGF-F | SEQ ID NO: 2 | GATTTGACCCTTAATGATGATGATGATGATGTCTCAATTC |
| EGF-R | SEQ ID NO: 3 | CTAATCTAAGAATTCTGATTCTGAATGTCCATTG |
| EGF-pY26-F | SEQ ID NO: 4 | GAATCAGAATTCTTAGATTAGATTGCTATGCTTTCTTTC |
| EGF-pY26-R | SEQ ID NO: 5 | CATCATCATCATCATCATTAAGGGTCAAATCGTTGGTAGATAC |
| bFGF-F | SEQ ID NO: 6 | CTAATCTAAGATGGCTGCTGGTTCTATTACTAC |
| bFGF-R | SEQ ID NO: 7 | GATTTGACCCTTAATGATGATGATGATGATGAGATTTAGC |
| bFGF-pY26-F | SEQ ID NO: 8 | CAGCAGCCATCTTAGATTAGATTGCTATGCTTTCTTTC |
| bFGF-pY26-R | SEQ ID NO: 9 | CATCATCATCATCATCATTAAGGGTCAAATCGTTGGTAGATAC |
| PDGF-BB-F | SEQ ID NO: 10 | CTAATCTAAGTCTTTGGGTTCTTTGACTATTGC |
| PDGF-BB-R | SEQ ID NO: 11 | GATTTGACCCTTAATGATGATGATGATGATGAGTAACAG |
| PDGF-BB-pY26-F | SEQ ID NO: 12 | GAACCCAAAGACTTAGATTAGATTGCTATGCTTTCTTTC |
| PDGF-BB-pY26-R | SEQ ID NO: 13 | CATCATCATCATCATCATTAAGGGTCAAATCGTTGGTAGATAC |
| LR³-IGF-1-F | SEQ ID NO: 14 | CTAATCTAAGATGTTTCCTGCTATGCCATTG |
| LR³-IGF-1-R | SEQ ID NO: 15 | GATTTGACCCTTAATGATGATGATGATGATGAGCAG |
| LR³-IGF-1-pY26-F | SEQ ID NO: 16 | CATCATCATCATCATCATTAAGGGTCAAATCGTTGGTAGATAC |
| LR³-IGF-1-pY26-R | SEQ ID NO: 17 | CAGGAAACATCTTAGATTAGATTGCTATGCTTTCTTTC |

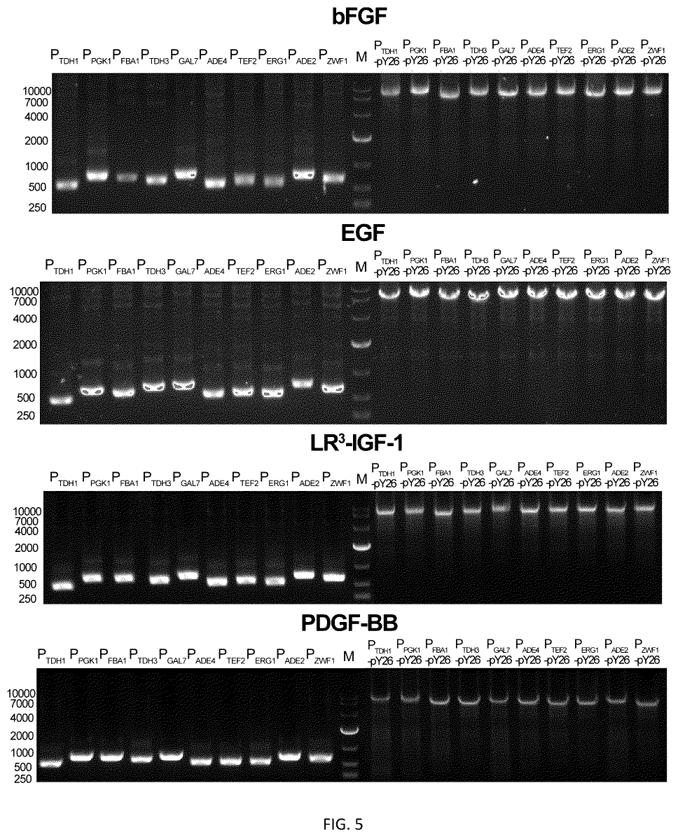

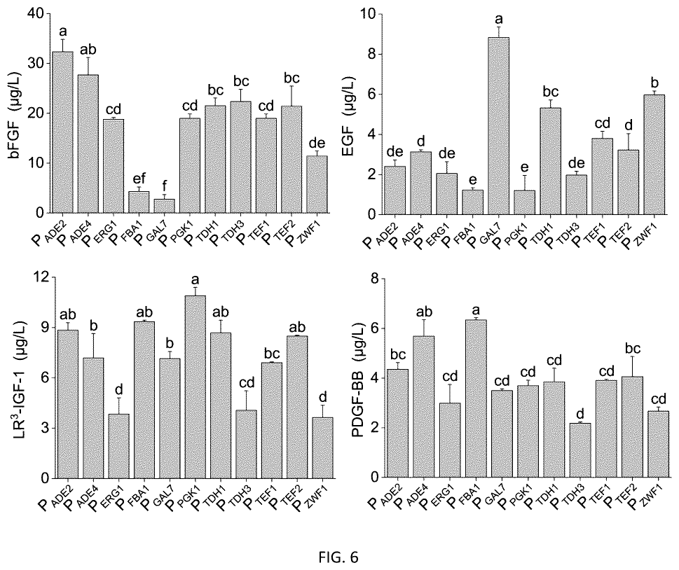

To further increase the expression levels of cytokines, the promoters of each cytokine were optimized. 10 promoters with different strengths (P_{TDH1}, P_{PGK1}, P_{TDH3}, P_{FBA1}, P_{GAL7}, P_{ADE4}, P_{TEF2}, P_{ERG1}, P_{ADE2}, and P_{ZWF1}) were selected to replace the original promoter P_{TEF1} on the pY26 plasmid, respectively. The pY26-Promoters-Cytokines plasmids were constructed using the yeast genome and pY26-Cytokine plasmid as templates by PCR with the primers in Table 2 to obtain the promoters and vector fragments, respectively (FIG. 5). The pY26-Promoters-Cytokines plasmids were transformed into S. cerevisiae C800. 5 positive strains for each plasmid were selected and fermented in a YPD medium in a shake flask for 2 d. After which, the bacterium was collected and fragmented to test the yields of different promoter groups. Elisa yield results showed that the optimal promoter combination differed for different cytokines. and the optimal promoters for bFGF, EGF, LR³-IGF-1, and PDGF-BB were P_{ADE2}, P_{GAL7}, P_{PGK1}, and P_{FBA1}, respectively. The optimal yields for the cytokines can be achieved with such promoters (FIG. 6).

TABLE 2
Primers for promoter optimization

| Name | SEQ ID NO: | Sequence (5′-3′) |
|---|---|---|
| EGF-TDH1-F | SEQ ID NO: 18 | GAATCAGAATTTTTGTTTTGTGTGTAAATTTAGTGAAGTACT |
| | | GTTTTTTGTG |
| EGF-TDH1-R | SEQ ID NO: 19 | CTTTGAAATGGCGAGTATTGATAATGAGAAACCACACCGTGG |
| | | GGC |
| EGF-TDH1-pY26-F | SEQ ID NO: 20 | CACAAAACAAAAATTCTGATTCTGAATGTCCATTGTCTCATG |
| EGF-TDH1-pY26-R | SEQ ID NO: 21 | CGGTGTGGTTTCTCATTATCAATACTCGCCATTTCAAAGAAT |
| | | ACG |
| EGF-PGK1-F | SEQ ID NO: 22 | GAATCAGAATTTGTTTTATATTTGTTGTAAAAAGTAGATAAT |
| | | TACTTCCTTGATGATCTG |
| EGF-PGK1-R | SEQ ID NO: 23 | GGCGAGTATTGATAATGAGTGAGTAAGGAAAGAGTGAGGAAC |
| | | TATCG |
| EGF-PGK1-pY26-F | SEQ ID NO: 24 | CAAATATAAAACAAATTCTGATTCTGAATGTCCATTGTCTCA |
| | | TG |
| EGF-PGK1-pY26-R | SEQ ID NO: 25 | CCTTACTCACTCATTATCAATACTCGCCATTTCAAAGAATAC |
| | | G |
| EGF-FBA1-F | SEQ ID NO: 26 | GAATCAGAATTTTTGAATATGTATTACTTGGTTATGGTTATA |
| | | TATGACAAAAGAAAAAG |
| EGF-FBA1-R | SEQ ID NO: 27 | GTATTGATAATGAATAACAATACTGACAGTACTAAATAATTG |
| | | CCTACTTGG |
| EGF-FBA1-pY26-F | SEQ ID NO: 28 | CCAAGTAATACATATTCAAAAATTCTGATTCTGAATGTCCAT |
| | | TGTCTCATG |
| EGF-FBA1-pY26-R | SEQ ID NO: 29 | GTACTGTCAGTATTGTTATTCATTATCAATACTCGCCATTTC |
| | | AAAGAATACG |
| EGF-TDH3-F | SEQ ID NO: 30 | CAGAATCAGAATTTTTGTTTGTTTATGTGTGTTTATTCGAAA |
| | | CTAAGTTCTTG |
| EGF-TDH3-R | SEQ ID NO: 31 | CCACTAGTTCTAGAATCCGATAAAAAACACGCTTTTTCAGTT |
| | | CGAGTTTATC |
| EGF-TDH3-pY26-F | SEQ ID NO: 32 | CATAAACAAACAAAAATTCTGATTCTGAATGTCCATTGTCTC |
| | | ATG |
| EGF-TDH3-pY26-R | SEQ ID NO: 33 | GAAAAAGCGTGTTTTTTATCGGATTCTAGAACTAGTGGATCC |
| | | CC |
| EGF-GAL7-F | SEQ ID NO: 34 | GAATCAGAATTTTTTGAGGGAATATTCAACTGTTTTTTTTTA |
| | | TCATGTTGATG |
| EGF-GAL7-R | SEQ ID NO: 35 | GGCGAGTATTGATAATGATTTGCCAGCTTACTATCCTTCTTG |
| | | AAAATATGC |
| EGF-GAL7-pY26-F | SEQ ID NO: 36 | GAATATTCCCTCAAAAAATTCTGATTCTGAATGTCCATTGTC |
| | | TCATG |
| EGF-GAL7-pY26-R | SEQ ID NO: 37 | GTAAGCTGGCAAATCATTATCAATACTCGCCATTTCAAAGAA |
| | | TACG |
| EGF-ADE4-F | SEQ ID NO: 38 | CAGAATCAGAATTTTTTTCTATTCTGCTGTTTGCTGTACCTC |
| | | TTTC |
| EGF-ADE4-R | SEQ ID NO: 39 | GGCGAGTATTGATAATGAGTCATTGTACCGCGAACTTTGGAC |
| EGF-ADE4-pY26-F | SEQ ID NO: 40 | GGTACAGCAAACAGCAGAATAGAAAAAAATTCTGATTCTGAA |
| | | TGTCCATTGTCTCATG |
| EGF-ADE4-pY26-R | SEQ ID NO: 41 | GGTACAATGACTCATTATCAATACTCGCCATTTCAAAGAATA |
| | | CGTAAATAATTAATAGTAG |
| EGF-TEF2-F | SEQ ID NO: 42 | CAGAATCAGAATTGTTTAGTTAATTATAGTTCGTTGACCGTA |
| | | TATTCTAAAAACAAG |
| EGF-TEF2-R | SEQ ID NO: 43 | GAAATGGCGAGTATTGATAATGAGGGGCCGTATACTTACATA |
| | | TAGTAGATGTC |
| EGF-TEF2-pY26-F | SEQ ID NO: 44 | CTATAATTAACTAAACAATTCTGATTCTGAATGTCCATTGTC |
| | | TCATG |
| EGF-TEF2-pY26-R | SEQ ID NO: 45 | GTATACGGCCCCTCATTATCAATACTCGCCATTTCAAAGAAT |
| | | ACG |
| EGF-ERG1-F | SEQ ID NO: 46 | CAGAATCAGAATTGACCCTTTTCTCGATATGTTTTTCTGTGA |
| | | TTTTTTTTTTTC |
| EGF-ERG1-R | SEQ ID NO: 47 | GAAATGGCGAGTATTGATAATGAGTGAATGGTATGAACATGG |
| | | ACATGAGC |
| EGF-ERG1-pY26-F | SEQ ID NO: 48 | CATATCGAGAAAAGGGTCAATTCTGATTCTGAATGTCCATTG |
| | | TCTCATG |
| EGF-ERG1-pY26-R | SEQ ID NO: 49 | GTTCATACCATTCACTCATTATCAATACTCGCCATTTCAAAG |
| | | AATACG |
| EGF-ADE2-F | SEQ ID NO: 50 | CAGAATCAGAATTACTTGATTGTTTTGTCCGATTTTCTTGTT |
| | | TTTC |
| EGF-ADE2-R | SEQ ID NO: 51 | GAGTATTGATAATGACTAGTAACGCCGTATCGTGATTAACG |
| EGF-ADE2-pY26-F | SEQ ID NO: 52 | CAAAACAATCAAGTAATTCTGATTCTGAATGTCCATTGTCTC |
| | | ATG |
| EGF-ADE2-pY26-R | SEQ ID NO: 53 | CGGCGTTACTAGTCATTATCAATACTCGCCATTTCAAAGAAT |
| | | ACG |
| EGF-ZWF1-F | SEQ ID NO: 54 | CAGAATCAGAATTCTTGCCTTATGTGGTTTTCTATTCTATTG |
| | | GATTTAC |
| EGF-ZWF1-R | SEQ ID NO: 55 | CTTTGAAATGGCGAGTATTGATAATGAGCCGTCGAAAAGGAT |
| | | CTCGTC |
| EGF-ZWF1-pY26-F | SEQ ID NO: 56 | CCACATAAGGCAAGAATTCTGATTCTGAATGTCCATTGTCTC |
| | | ATG |
| EGF-ZWF1-pY26-R | SEQ ID NO: 57 | CCTTTTCGACGGCTCATTATCAATACTCGCCATTTCAAAGAA |
| | | TACG |
| bFGF-TDH1-F | SEQ ID NO: 58 | CAGCAGCCATTTTGTTTTGTGTGTAAATTTAGTGAAGTACTG |
| | | TTTTTTGTG |
| bFGF-TDH1-R | SEQ ID NO: 59 | GAAATGGCGAGTATTGATAATGAGAAACCACACCGTGGGGC |
| bFGF-TDH1-pY26-F | SEQ ID NO: 60 | CACAAAACAAAATGGCTGCTGGTTCTATTACTACTTTGC |
| bFGF-TDH1-pY26-R | SEQ ID NO: 61 | GTGTGGTTTCTCATTATCAATACTCGCCATTTCAAAGAATAC |
| | | G |
| bFGF-PGK1-F | SEQ ID NO: 62 | CAGCAGCCATTGTTTTATATTTGTTGTAAAAAGTAGATAATT |
| | | ACTTCCTTGATGATCTG |
| bFGF-PGK1-R | SEQ ID NO: 63 | GAAATGGCGAGTATTGATAATGAGTGAGTAAGGAAAGAGTGA |
| | | GGAACTATCG |
| bFGF-PGK1-pY26-F | SEQ ID NO: 64 | CAAATATAAAACAATGGCTGCTGGTTCTATTACTACTTTGC |
| bFGF-PGK1-pY26-R | SEQ ID NO: 65 | CCTTACTCACTCATTATCAATACTCGCCATTTCAAAGAATAC |
| | | G |
| bFGF-TDH3-F | SEQ ID NO: 66 | CAGCAGCCATTTTGTTTGTTTATGTGTGTTTATTCGAAACTA |
| | | AGTTCTTG |
| bFGF-TDH3-R | SEQ ID NO: 67 | GTTCTAGAATCCGATAAAAAACACGCTTTTTCAGTTCGAGTT |
| | | TATC |
| bFGF-TDH3-pY26-F | SEQ ID NO: 68 | CATAAACAAACAAAATGGCTGCTGGTTCTATTACTACTTTGC |
| bFGF-TDH3-pY26-R | SEQ ID NO: 69 | GCGTGTTTTTTATCGGATTCTAGAACTAGTGGATCCCC |
| bFGF-FBA1-F | SEQ ID NO: 70 | CAGCAGCCATTTTGAATATGTATTACTTGGTTATGGTTATAT |
| | | ATGACAAAAGAAAAAG |
| bFGF-FBA1-R | SEQ ID NO: 71 | GTATTGATAATGAATAACAATACTGACAGTACTAAATAATTG |
| | | CCTACTTGG |
| bFGF-FBA1-pY26-F | SEQ ID NO: 72 | CATATTCAAAATGGCTGCTGGTTCTATTACTACTTTGC |
| bFGF-FBA1-pY26-R | SEQ ID NO: 73 | GTATTGTTATTCATTATCAATACTCGCCATTTCAAAGAATAC |
| | | G |
| bFGF-GAL7-F | SEQ ID NO: 74 | CCAGCAGCCATTTTTGAGGGAATATTCAACTGTTTTTTTTTA |
| | | TCATGTTGATG |
| bFGF-GAL7-R | SEQ ID NO: 75 | GAGTATTGATAATGATTTGCCAGCTTACTATCCTTCTTGAAA |
| | | ATATG |
| bFGF-GAL7-pY26-F | SEQ ID NO: 76 | GAATATTCCCTCAAAAATGGCTGCTGGTTCTATTACTACTTT |
| | | GC |
| bFGF-GAL7-pY26-R | SEQ ID NO: 77 | GTAAGCTGGCAAATCATTATCAATACTCGCCATTTCAAAGAA |
| | | TACG |
| bFGF-ADE4-F | SEQ ID NO: 78 | CAGCAGCCATTTTTTCTATTCTGCTGTTTGCTGTACCTCTTT |
| bFGF-ADE4-R | SEQ ID NO: 79 | CGTATTGATAATGAGTCATTGTACCGCGAACTTTGGAC |
| bFGF-ADE4-pY26-F | SEQ ID NO: 80 | GAATAGAAAAAATGGCTGCTGGTTCTATTACTACTTTGC |
| bFGF-ADE4-pY26-R | SEQ ID NO: 81 | GTACAATGACTCATTATCAATACTCGCCATTTCAAAGAATAC |
| | | G |
| bFGF-TEF2-F | SEQ ID NO: 82 | CCAGCAGCCATGTTTAGTTAATTATAGTTCGTTGACCGTATA |
| | | TTCTAAAAACAAG |
| bFGF-TEF2-R | SEQ ID NO: 83 | GAGTATTGATAATGAGGGGCCGTATACTTACATATAGTAGAT |
| | | GTC |
| bFGF-TEF2-pY26-F | SEQ ID NO: 84 | CTATAATTAACTAAACATGGCTGCTGGTTCTATTACTACTTT |
| | | GC |
| bFGF-TEF2-pY26-R | SEQ ID NO: 85 | GTATACGGCCCCTCATTATCAATACTCGCCATTTCAAAGAAT |
| | | ACG |
| bFGF-ERG1-F | SEQ ID NO: 86 | CAGCAGCCATGACCCTTTTCTCGATATGTTTTTCTGTGATTT |
| | | TTTTTTTTC |
| bFGF-ERG1-R | SEQ ID NO: 87 | GCGAGTATTGATAATGAGTGAATGGTATGAACATGGACATGA |
| | | GC |
| bFGF-ERG1-pY26-F | SEQ ID NO: 88 | GAAAAGGGTCATGGCTGCTGGTTCTATTACTACTTTGC |
| bFGF-ERG1-pY26-R | SEQ ID NO: 89 | CATACCATTCACTCATTATCAATACTCGCCATTTCAAAGAAT |
| | | ACG |
| bFGF-ADE2-F | SEQ ID NO: 90 | CCAGCAGCCATACTTGATTGTTTTGTCCGATTTTCTTGTTTT |
| | | TCTTG |
| bFGF-ADE2-R | SEQ ID NO: 91 | GTATTGATAATGACTAGTAACGCCGTATCGTGATTAACGTAT |
| | | TAC |
| bFGF-ADE2-pY26-F | SEQ ID NO: 92 | CAAAACAATCAAGTATGGCTGCTGGTTCTATTACTACTTTGC |
| bFGF-ADE2-pY26-R | SEQ ID NO: 93 | GGCGTTACTAGTCATTATCAATACTCGCCATTTCAAAGAATA |
| | | CG |
| bFGF-ZWF1-F | SEQ ID NO: 94 | CCAGCAGCCATCTTGCCTTATGTGGTTTTCTATTCTATTGGA |
| | | TTTACTC |
| bFGF-ZWF1-R | SEQ ID NO: 95 | GTATTGATAATGAGCCGTCGAAAAGGATCTCGTC |
| bFGF-ZWF1-pY26-F | SEQ ID NO: 96 | CATAAGGCAAGATGGCTGCTGGTTCTATTACTACTTTGC |
| bFGF-ZWF1-pY26-R | SEQ ID NO: 97 | CTTTTCGACGGCTCATTATCAATACTCGCCATTTCAAAGAAT |
| | | ACG |
| PDGF-TDH1-F | SEQ ID NO: 98 | GAACCCAAAGATTTGTTTTGTGTGTAAATTTAGTGAAGTACT |
| | | GTTTTTTG |
| PDGF-TDH1-R | SEQ ID NO: 99 | GGCGAGTATTGATAATGAGAAACCACACCGTGGGGC |
| PDGF-TDH1-pY26-F | SEQ ID NO: | CACACAAAACAAATCTTTGGGTTCTTTGACTATTGCTGAACC |
| | 100 | |
| PDGF-TDH1-pY26-R | SEQ ID NO: | CGGTGTGGTTTCTCATTATCAATACTCGCCATTTCAAAGAAT |
| | 101 | ACG |
| PDGF-PGK1-F | SEQ ID NO: | GAACCCAAAGATGTTTTATATTTGTTGTAAAAAGTAGATAAT |
| | 102 | TACTTCCTTGATGATCTG |
| PDGF-PGK1-R | SEQ ID NO: | CTTTGAAATGGCGAGTATTGATAATGAGTGAGTAAGGAAAGA |
| | 103 | GTGAGGAACTATCG |
| PDGF-PGK1-pY26-F | SEQ ID NO: | CAAATATAAAACATCTTTGGGTTCTTTGACTATTGCTGAACC |
| | 104 | |
| PDGF-PGK1-pY26-R | SEQ ID NO: | CCTTACTCACTCATTATCAATACTCGCCATTTCAAAGAATAC |
| | 105 | G |
| PDGF-TDH3-F | SEQ ID NO: | CAAAGAACCCAAAGATTTGTTTGTTTATGTGTGTTTATTCGA |
| | 106 | AACTAAGTTCTTG |
| PDGF-TDH3-R | SEQ ID NO: | CTAGTTCTAGAATCCGATAAAAAACACGCTTTTTCAGTTCGA |
| | 107 | GTTTATCATTATC |
| PDGF-TDH3-pY26-F | SEQ ID NO: | CACACATAAACAAACAAATCTTTGGGTTCTTTGACTATTGCT |
| | 108 | GAACC |
| PDGF-TDH3-pY26-R | SEQ ID NO: | CTGAAAAAGCGTGTTTTTTATCGGATTCTAGAACTAGTGGAT |
| | 109 | CCCC |
| PDGF-FBA1-F | SEQ ID NO: | GAACCCAAAGATTTGAATATGTATTACTTGGTTATGGTTATA |
| | 110 | TATGACAAAAGAAAAAG |
| PDGF-FBA1-R | SEQ ID NO: | GGCGAGTATTGATAATGAATAACAATACTGACAGTACTAAAT |
| | 111 | AATTGCCTACTTGG |
| PDGF-FBA1-pY26-F | SEQ ID NO: | GTAATACATATTCAAATCTTTGGGTTCTTTGACTATTGCTGA |
| | 112 | ACC |
| PDGF-FBA1-pY26-R | SEQ ID NO: | CAGTATTGTTATTCATTATCAATACTCGCCATTTCAAAGAAT |
| | 113 | ACG |
| PDGF-GAL7-F | SEQ ID NO: | CAAAGAACCCAAAGATTTTGAGGGAATATTCAACTGTTTTTT |
| | 114 | TTTATCATGTTG |
| PDGF-GAL7-R | SEQ ID NO: | GGCGAGTATTGATAATGATTTGCCAGCTTACTATCCTTCTTG |
| | 115 | AAAATATG |
| PDGF-GAL7-pY26-F | SEQ ID NO: | CAGTTGAATATTCCCTCAAAATCTTTGGGTTCTTTGACTATT |
| | 116 | GCTGAACC |
| PDGF-GAL7-pY26-R | SEQ ID NO: | GATAGTAAGCTGGCAAATCATTATCAATACTCGCCATTTCAA |
| | 117 | AGAATACG |
| PDGF-ADE4-F | SEQ ID NO: | GTCAAAGAACCCAAAGATTTTTCTATTCTGCTGTTTGCTGTA |
| | 118 | CCTC |
| PDGF-ADE4-R | SEQ ID NO: | GGCGAGTATTGATAATGAGTCATTGTACCGCGAACTTTGGAC |
| | 119 | |
| PDGF-ADE4-pY26-F | SEQ ID NO: | CAGCAGAATAGAAAAATCTTTGGGTTCTTTGACTATTGCTGA |
| | 120 | ACC |
| PDGF-ADE4-pY26-R | SEQ ID NO: | CGGTACAATGACTCATTATCAATACTCGCCATTTCAAAGAAT |
| | 121 | ACG |
| PDGF-TEF2-F | SEQ ID NO: | GAACCCAAAGAGTTTAGTTAATTATAGTTCGTTGACCGTATA |
| | 122 | TTCTAAAAACAAG |
| PDGF-TEF2-R | SEQ ID NO: | CGAGTATTGATAATGAGGGGCCGTATACTTACATATAGTAGA |
| | 123 | TGTCAAG |
| PDGF-TEF2-pY26-F | SEQ ID NO: | CTATAATTAACTAAACTCTTTGGGTTCTTTGACTATTGCTGA |
| | 124 | ACC |
| PDGF-TEF2-pY26-R | SEQ ID NO: | GTATACGGCCCCTCATTATCAATACTCGCCATTTCAAAGAAT |
| | 125 | ACG |
| PDGF-ERG1-F | SEQ ID NO: | CAAAGAACCCAAAGAGACCCTTTTCTCGATATGTTTTTCTGT |
| | 126 | GATTTTTTTTTTTC |
| PDGF-ERG1-R | SEQ ID NO: | GGCGAGTATTGATAATGAGTGAATGGTATGAACATGGACATG |
| | 127 | AGC |
| PDGF-ERG1-pY26-F | SEQ ID NO: | CGAGAAAAGGGTCTCTTTGGGTTCTTTGACTATTGCTGAACC |
| | 128 | |
| PDGF-ERG1-pY26-R | SEQ ID NO: | CATACCATTCACTCATTATCAATACTCGCCATTTCAAAGAAT |
| | 129 | ACG |
| PDGF-ADE2-F | SEQ ID NO: | CAAAGAACCCAAAGAACTTGATTGTTTTGTCCGATTTTCTTG |
| | 130 | TTTTTC |
| PDGF-ADE2-R | SEQ ID NO: | CGAGTATTGATAATGACTAGTAACGCCGTATCGTGATTAACG |
| | 131 | TATTAC |
| PDGF-ADE2-pY26-F | SEQ ID NO: | CAAAACAATCAAGTTCTTTGGGTTCTTTGACTATTGCTGAAC |
| | 132 | C |
| PDGF-ADE2-pY26-R | SEQ ID NO: | GCGTTACTAGTCATTATCAATACTCGCCATTTCAAAGAATAC |
| | 133 | G |
| PDGF-ZWF1-F | SEQ ID NO: | CAAAGAACCCAAAGACTTGCCTTATGTGGTTTTCTATTCTAT |
| | 134 | TGGATTTAC |
| PDGF-ZWF1-R | SEQ ID NO: | GAAATGGCGAGTATTGATAATGAGCCGTCGAAAAGGATCTCG |
| | 135 | TC |
| PDGF-ZWF1-pY26-F | SEQ ID NO: | CCACATAAGGCAAGTCTTTGGGTTCTTTGACTATTGCTGAAC |
| | 136 | C |
| PDGF-ZWF1-pY26-R | SEQ ID NO: | CTTTTCGACGGCTCATTATCAATACTCGCCATTTCAAAGAAT |
| | 137 | ACG |
| IGF-1-TDH1-F | SEQ ID NO: | GCAGGAAACATTTTGTTTTGTGTGTAAATTTAGTGAAGTACT |
| | 138 | GTTTTTTG |
| IGF-1-TDH1-R | SEQ ID NO: | CTTTGAAATGGCGAGTATTGATAATGAGAAACCACACCGTGG |
| | 139 | GGC |
| IGF-1-TDH1-pY26-F | SEQ ID NO: | CACACAAAACAAAATGTTTCCTGCTATGCCATTGTCTTC |
| | 140 | |
| IGF-1-TDH1-pY26-R | SEQ ID NO: | CGGTGTGGTTTCTCATTATCAATACTCGCCATTTCAAAGAAT |
| | 141 | ACG |
| IGF-1-PGK1-F | SEQ ID NO: | GCAGGAAACATTGTTTTATATTTGTTGTAAAAAGTAGATAAT |
| | 142 | TACTTCCTTGATGATCTG |
| IGF-1-PGK1-R | SEQ ID NO: | GAAATGGCGAGTATTGATAATGAGTGAGTAAGGAAAGAGTGA |
| | 143 | GGAACTATCG |
| IGF-1-PGK1-pY26-F | SEQ ID NO: | CTTTTTACAACAAATATAAAACAATGTTTCCTGCTATGCCAT |
| | 144 | TGTCTTC |
| IGF-1-PGK1-pY26-R | SEQ ID NO: | CTTTCCTTACTCACTCATTATCAATACTCGCCATTTCAAAGA |
| | 145 | ATACG |
| IGF-1-TDH3-F | SEQ ID NO: | GCAGGAAACATTTTGTTTGTTTATGTGTGTTTATTCGAAACT |
| | 146 | AAGTTCTTG |
| IGF-1-TDH3-R | SEQ ID NO: | CACTAGTTCTAGAATCCGATAAAAAACACGCTTTTTCAGTTC |
| | 147 | GAGTTTATC |
| IGF-1-TDH3-pY26-F | SEQ ID NO: | CATAAACAAACAAAATGTTTCCTGCTATGCCATTGTCTTC |
| | 148 | |
| IGF-1-TDH3-pY26-R | SEQ ID NO: | GCGTGTTTTTTATCGGATTCTAGAACTAGTGGATCCCC |
| | 149 | |
| IGF-1-FBA1-F | SEQ ID NO: | GCAGGAAACATTTTGAATATGTATTACTTGGTTATGGTTATA |
| | 150 | TATGACAAAAGAAAAAG |
| IGF-1-FBA1-R | SEQ ID NO: | GGCGAGTATTGATAATGAATAACAATACTGACAGTACTAAAT |
| | 151 | AATTGCCTACTTGG |
| IGF-1-FBA1-pY26-F | SEQ ID NO: | CAAGTAATACATATTCAAAATGTTTCCTGCTATGCCATTGTC |
| | 152 | TTC |
| IGF-1-FBA1-pY26-R | SEQ ID NO: | CAGTATTGTTATTCATTATCAATACTCGCCATTTCAAAGAAT |
| | 153 | ACG |
| IGF-1-GAL7-F | SEQ ID NO: | CATAGCAGGAAACATTTTTGAGGGAATATTCAACTGTTTTTT |
| | 154 | TTTATCATGTTGATG |
| IGF-1-GAL7-R | SEQ ID NO: | GGCGAGTATTGATAATGATTTGCCAGCTTACTATCCTTCTTG |
| | 155 | AAAATATG |
| IGF-1-GAL7-pY26-F | SEQ ID NO: | GAATATTCCCTCAAAAATGTTTCCTGCTATGCCATTGTCTTC |
| | 156 | |
| IGF-1-GAL7-pY26-R | SEQ ID NO: | GTAAGCTGGCAAATCATTATCAATACTCGCCATTTCAAAGAA |
| | 157 | TACG |
| IGF-1-ADE4-F | SEQ ID NO: | CATAGCAGGAAACATTTTTTCTATTCTGCTGTTTGCTGTACC |
| | 158 | TC |
| IGF-1-ADE4-R | SEQ ID NO: | GGCGAGTATTGATAATGAGTCATTGTACCGCGAACTTTGGAC |
| | 159 | |
| IGF-1-ADE4-pY26-F | SEQ ID NO: | GCAGAATAGAAAAAATGTTTCCTGCTATGCCATTGTCTTC |
| | 160 | |
| IGF-1-ADE4-pY26-R | SEQ ID NO: | CGGTACAATGACTCATTATCAATACTCGCCATTTCAAAGAAT |
| | 161 | ACG |
| IGF-1-TEF2-F | SEQ ID NO: | GCATAGCAGGAAACATGTTTAGTTAATTATAGTTCGTTGACC |
| | 162 | GTATATTCTAAAAACAAG |
| IGF-1-TEF2-R | SEQ ID NO: | GAAATGGCGAGTATTGATAATGAGGGGCCGTATACTTACATA |
| | 163 | TAGTAGATGTC |
| IGF-1-TEF2-pY26-F | SEQ ID NO: | CTATAATTAACTAAACATGTTTCCTGCTATGCCATTGTCTTC |
| | 164 | |
| IGF-1-TEF2-pY26-R | SEQ ID NO: | GTATACGGCCCCTCATTATCAATACTCGCCATTTCAAAGAAT |
| | 165 | ACG |
| IGF-1-ERG1-F | SEQ ID NO: | CATAGCAGGAAACATGACCCTTTTCTCGATATGTTTTTCTGT |
| | 166 | GATTTTTTTTTTTC |
| IGF-1-ERG1-R | SEQ ID NO: | GAAATGGCGAGTATTGATAATGAGTGAATGGTATGAACATGG |
| | 167 | ACATGAGC |
| IGF-1-ERG1-pY26-F | SEQ ID NO: | GAAAAACATATCGAGAAAAGGGTCATGTTTCCTGCTATGCCA |
| | 168 | TTGTCTTC |
| IGF-1-ERG1-pY26-R | SEQ ID NO: | CATACCATTCACTCATTATCAATACTCGCCATTTCAAAGAAT |
| | 169 | ACGTAAATAATTAATAG |
| IGF-1-ADE2-F | SEQ ID NO: | CATAGCAGGAAACATACTTGATTGTTTTGTCCGATTTTCTTG |
| | 170 | TTTTTC |
| IGF-1-ADE2-R | SEQ ID NO: | GGCGAGTATTGATAATGACTAGTAACGCCGTATCGTGATTAA |
| | 171 | CG |
| IGF-1-ADE2-pY26-F | SEQ ID NO: | CAAGAAAATCGGACAAAACAATCAAGTATGTTTCCTGCTATG |
| | 172 | CCATTGTCTTC |
| IGF-1-ADE2-pY26-R | SEQ ID NO: | CGGCGTTACTAGTCATTATCAATACTCGCCATTTCAAAGAAT |
| | 173 | ACGTAAATAATTAATAG |
| IGF-1-ZWF1-F | SEQ ID NO: | CATAGCAGGAAACATCTTGCCTTATGTGGTTTTCTATTCTAT |
| | 174 | TGGATTTAC |
| IGF-1-ZWF1-R | SEQ ID NO: | GAAATGGCGAGTATTGATAATGAGCCGTCGAAAAGGATCTCG |
| | 175 | TC |
| IGF-1-ZWF1-pY26-F | SEQ ID NO: | GAAAACCACATAAGGCAAGATGTTTCCTGCTATGCCATTGTC |
| | 176 | TTC |
| IGF-1-ZWF1-pY26-R | SEQ ID NO: | CCTTTTCGACGGCTCATTATCAATACTCGCCATTTCAAAGAA |
| | 177 | TACG |

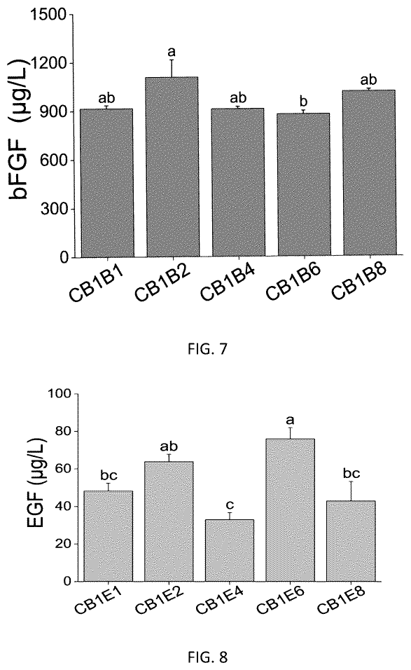

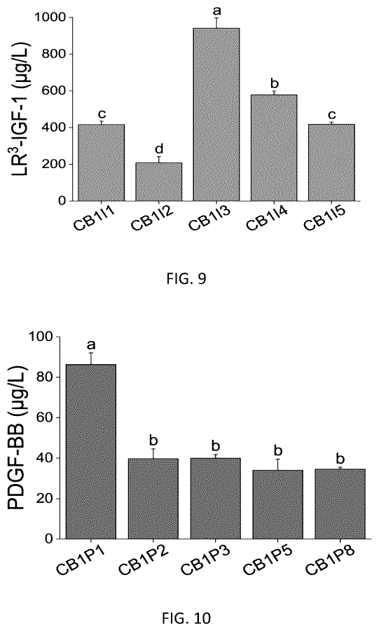

Four cytokines bFGF, EGF, LR³-IGF-1, and PDGF-BB with their respective optimal promoters were integrated into the S. cerevisiae C800 genome at the multi-copy locus Ty1, respectively, and the high-copy strains were screened using a TRP degradation tag. For the four cytokines, 5 positive strains that were the first to grow on the SD-TRP plates were selected and cultured in a YPD medium for 3 d. After which, the four cytokines were detected for yields using an ELISA kit, respectively. ELISA assay results showed that the cytokines were stably and highly expressed in the yeast genome, and the yields were about 7.22 to 86.44 times higher than that of the plasmid expression. The strains expressing the cytokines bFGF, EGF, LR³-IGF-1, and PDGF-BB at the highest yields were CB1B2, CB1E6, CB1I3, and CB1P1, respectively (FIGS. 7-10). A stable expression system of a single cytokine in S. cerevisiae was successfully achieved.

### Example 2: Construction of Chassis Strain Increasing Intracellular Expression of Proteins in Yeast

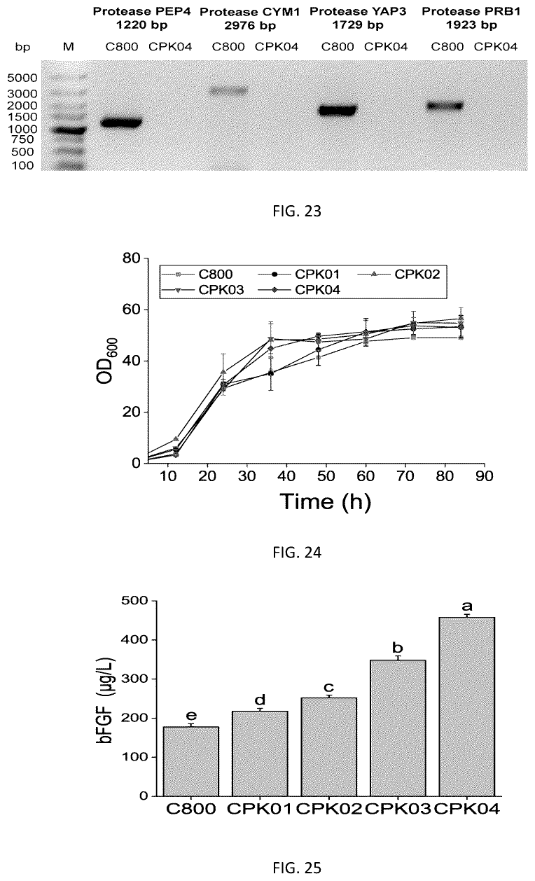

Recombinant cytokines were intracellularly expressed in S. cerevisiae, and endogenous proteases in yeast could hydrolyze the cytokines as generated. Therefore, endogenous proteases: PEP4, YAP3, PRB1 and CYM1 were selected for knockout in S. cerevisiae C800 to compare the effects on cytokine yields. A Cre-LoxP system was utilized to design knockout of endogenous proteases. The endogenous proteases PEP4, CYM1, YAP3, and PRB1 were knocked out sequentially, and the knockout primers were shown in Table 3. After verification of the correctness, a protease PEP4 single-deletion strain (CPK01), a proteases PEP4, YAP3 double-deletion strain (CPK02), a proteases PEP4, YAP3, PRB1 triple-deletion strain (CPK03), and a proteases PEP4, YAP3, PRB1 and CYM1 quadruple-deletion strain (CPK04) were obtained. Validation of the protease gene fragments for the original strain C800 and the proteases quadruple-knockout strain CPK04 showed that all of the four endogenous proteases in the CPK04 strain had been successfully knocked out (FIG. 23). In order to investigate the effect of protease knockout on the growth and cytokine yields of yeast strains, the cytokine bFGF and its optimal promoter P_{ADE2} were ligated to the screening tag URA3 gene to construct a P_{ADE2}-bFGF-URA3 gene fragment, which was inserted into the C800, CPK01, CPK02, CPK03, and CPK04 strains at the same single-copy locus, respectively. Screening was carried out using a URA-deficient YNB plate. The positive strains obtained from screening were fermented in 250 mL shake flasks to investigate the growth of different strains and the yields of bFGF in different strains, respectively. The results showed that the knockout of endogenous protease in yeast did not affect cell growth (FIG. 24). With the knockout of endogenous protease, the yields of bFGF gradually increased, and the CPK04 strain achieved the highest yield, which was about 2.58 times higher than that of the original strain (FIG. 25). It can be seen that the knockout of endogenous proteases in yeast facilitates the reduction of intracellular degradation of cytokines and facilitates the expression and accumulation of cytokines. Therefore, a protease-deficient S. cerevisiae chassis strain was successfully obtained, which can effectively increase the yield of heterologously expressed cytokines.

TABLE 3
Primers for protease knockout

| Name | SEQ ID NO: | Sequence (5′-3′) |
|---|---|---|
| CYM1-U-F | SEQ ID NO: 178 | GGGTCTTCTAATGCTGAAAGTTCAC |
| CYM1-U-R | SEQ ID NO: 179 | ATCCGCTCACAATTCCACAATATATAATTTAATCCTGTAAATATAC |
| | | TGCTTCAAAGTTTC |
| CYM1-TRP-F | SEQ ID NO: 180 | AGCAGTATATTTACAGGATTAAATTATATATTGTGGAATTGTGAGC |
| | | GGATAACAATTTC |
| CYM1-TRP-R | SEQ ID NO: 181 | AAGTTACTATAAAATATACTGCGGTATATATGCGTAAGGAGAAAAT |
| | | ACCGCATC |
| CYM1-D-F | SEQ ID NO: 182 | CGGTATTTTCTCCTTACGCATATATACCGCAGTATATTTTATAGTA |
| | | ACTTTATTCTTTTG |
| CYM1-D-F | SEQ ID NO: 183 | CGGTATTTTCTCCTTACGCATATATACCGCAGTATATTTTATAGTA |
| | | ACTTTATTCTTTTG |
| PEP4-U-F | SEQ ID NO: 184 | GAAGGGATCGGATTTGGCTG |
| PEP4-U-R | SEQ ID NO: 185 | GTAATCATGGTCATAGCTGTTTCCTGGCTAAACTTTTCTTACTTCT |
| | | CCGC |
| PEP4-LEU-F | SEQ ID NO: 186 | GCGGAGAAGTAAGAAAAGTTTAGCCAGGAAACAGCTATGACCATGA |
| | | TTACGC |
| PEP4-LEU-R | SEQ ID NO: 187 | CAAATAAAATTCAAACAAAAACCAAAACTAACTAAAACGACGGCCA |
| | | GTGCC |
| PEP4-D-F | SEQ ID NO: 188 | GGCACTGGCCGTCGTTTTAGTTAGTTTTGGTTTTTGTTTGAATTTT |
| | | ATTTGG |
| PEP4-D-R | SEQ ID NO: 189 | GTTATGCTTAATGAATTTTTGAATCAGAAAAAGAAG |
| PRB1-U-F | SEQ ID NO: 190 | GGAACAGTGTGACATCCAACC |
| PRB1-U-R | SEQ ID NO: 191 | CATGGTCATAGCTGTTTCCTGTTCTTCATTTAGAAAAATTTCAGCT |
| | | GCTTTTTTTTTTC |
| PRB1-LEU-F | SEQ ID NO: 192 | AAGCAGCTGAAATTTTTCTAAATGAAGAACAGGAAACAGCTATGAC |
| | | CATGATTACG |
| PRB1-LEU-R | SEQ ID NO: 193 | TAAAAAAACAAACTAAACCTAATTCTAACAAGCAAAGTAAAACGAC |
| | | GGCCAGTGCCAA |
| PRB1-D-F | SEQ ID NO: 194 | CACTGGCCGTCGTTTTACTTTGCTTGTTAGAATTAGGTTTAGTTTG |
| | | TTTTTTTATTGG |
| PRB1-D-R | SEQ ID NO: 195 | CAACTGCCGGCTGAAAGAGC |
| YAP3-U-F | SEQ ID NO: 196 | CATGGCCTAAGTATTGTGAAGTAATCAAATC |
| YAP3-U-R | SEQ ID NO: 197 | CATGGTCATAGCTGTTTCCTGATAGACTTTGACAAAAATAACTTAC |
| | | AAACACTAAATAAG |
| YAP3-HIS-F | SEQ ID NO: 198 | GTTTGTAAGTTATTTTTGTCAAAGTCTATCAGGAAACAGCTATGAC |
| | | CATGATTAC |
| YAP3-HIS-R | SEQ ID NO: 199 | CTAACGAAGAATAAGGCTGAAGCAATTGTTTAAAACGACGGCCAGT |
| | | GC |
| YAP3-D-F | SEQ ID NO: 200 | TGGCACTGGCCGTCGTTTTAAACAATTGCTTCAGCCTTATTCTTCG |
| | | TTAG |
| YAP3-D-R | SEQ ID NO: 201 | CATTCCTTCTGCAATAGGCGCAATC |

### Example 3: Co-Expression of Multiple Cytokines in S. cerevisiae

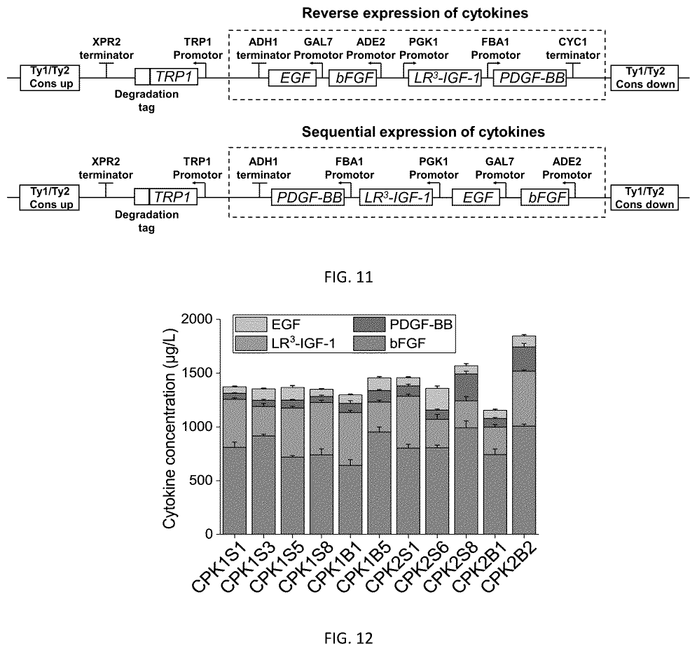

To achieve the co-expression of four cytokines bFGF, EGF, LR³-IGF-1 and PDGF-BB in the protease-deficient strain CPK04 in Example 2, a cytokine co-expression cassette was constructed. The promoters in the expression elements of the four cytokine-encoding genes were the optimal promoters obtained in Example 1, and the cytokines were combined sequentially or concurrently, respectively (FIG. 11). The multi-copy loci Ty1 and Ty2 on the yeast genome were selected for integration. In order to further increase the expression level of the cytokines, the multi-copy strains were screened utilizing TRP1 with degradation tags, and the strains that were the first to grow on a TRP-deficient YNB plate possessed more copies of the fragments. For each cytokine, 5 positive strains were selected for fermentation, and the bacterium was fragmented. After which, the yields of each cytokine were measured.

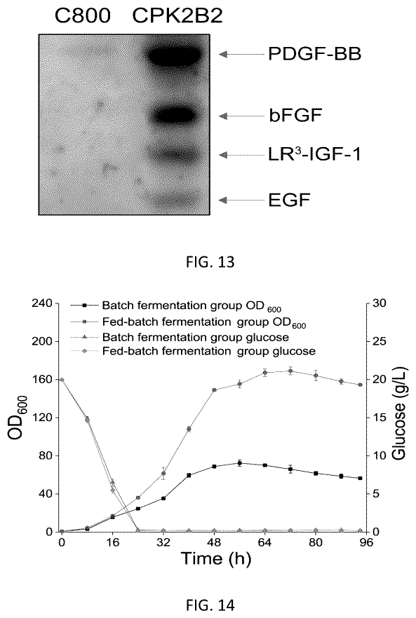

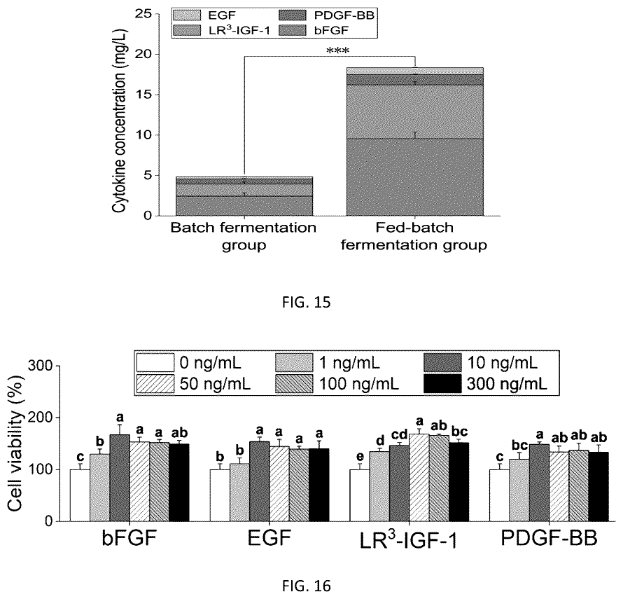

The assay results showed that the strain CPK2B2 integrated at the multi-copy locus Ty2 achieved the highest cytokine yield of 1845.67 μg/L (FIG. 12). It can be seen that the 4 cytokines were best expressed when the order of expression was concurrent and when bFGF and EGF were grouped together and LR³-IGF-1 and PDGF-BB were grouped together. 6*His Tag-specific WB results showed that the CPK2B2 strain could co-express four cytokines (FIG. 13). The disclosure successfully achieved the high expression of 4 cytokines within the same strain. Since the cytokine yield was positively correlated with the amount of yeast, it was improved by increasing the amount of yeast through a high-density fermentation in a 3 L fermenter. The fermentation process was also optimized to provide sufficient glucose and free amino acids for rapid yeast growth and cytokine synthesis. Specifically, when the initial glucose was depleted (at about 20 h), a fed-batch fermentation was started and terminated after 62 h. Fermentation was stopped at 94 h when the yeast stopped growing, and the OD₆₀₀ of yeast cells in the fed-batch fermentation group could reach about 160, which was 2.53 times higher than that of the batch fermentation group (FIG. 14). Detection of cytokine yields showed that the total cytokine yield after the fed-batch fermentation in a 3 L fermenter could reach 18.35 mg/L, which was 3.78 times higher than that of the batch fermentation control group, and 529 times higher than that of the free plasmid expression (FIG. 15). The construction of a stable expression system of several cytokines in S. cerevisiae was successfully achieved. Among them, the yields of bFGF, EGF and PDGF-BB, and LR³-IGF-1 reached 9.56 mg/L, 0.87 mg/L, 1.28 mg/L, and 6.64 mg/L, respectively.

### Example 4: Physical Fragmentation Protocol for Recombinant S. cerevisiae Expressing Cytokines Intracellularly

In order to minimize costs, complex and costly purification processes shall be avoided. A physical fragmentation protocol was developed for recombinant S. cerevisiae expressing cytokines. After subjecting to a physical fragmentation using an instrument such as FastPrep-24™ or a high-pressure homogenizer, the cytokines had normal biological activity and could be directly applied to seed cell culture.

Specifically, the recombinant S. cerevisiae was inoculated in a 3 L fermenter containing a YPD medium. When the initial glucose was depleted, a fed-batch fermentation was carried out at a rate of 5 mL/h and terminated after 62 h. The fermentation was terminated when the bacterium was no longer growing, and the wet yeast bacterium was harvested. The wet bacterium as obtained was carefully washed 2 times using PBS to remove medium residues. The bacterium was washed and resuspended again with PBS, and then a high-pressure homogenizer was used for batch fragmentation of wet yeast bacterium under the pressure of 1000-1400 bar for 4-6 cycles. The temperature of the high-pressure homogenizer was set as 4° C. to avoid the denaturation of cytokines by high temperatures. After fragmentation, the fragmented broth was collected and centrifuged at 4° C. and 8000 rpm for 10 min, and the bacterial fragments were removed to obtain a yeast lysate. The endotoxins were removed. After which, a mother liquor of the sterile yeast lysate was obtained with filtration and sterilization, diluted to a suitable ratio and then added into a cell medium for cell culture.

### Example 5: Application of Recombinant S. cerevisiae Lysate Co-Expressing Four Cytokines in Cell-Cultured Meat Production

The optimal concentration of four cytokines bFGF, EGF, LR³-IGF-1 and PDGF-BB for promoting the proliferation of muscle stem cells was firstly investigated. The four cytokines were added to a DMEM medium containing 5% FBS at 1 ng/mL, 10 ng/mL, 50 ng/mL, 100 ng/mL and 300 ng/mL to culture the muscle stem cells, respectively, and the activity of the cells in each group was measured after 2 d. The results showed that the optimal concentration of bFGF, EGF and PDGF-BB for promoting the cell proliferation was 10 ng/mL, and the optimal concentration of LR³-IGF-1 for promoting the cell proliferation was 50 ng/mL (FIG. 16). 10 ng/mL of bFGF, EGF, and PDGF-BB and 50 ng/mL of LR³-IGF-1 were used as standard samples for commercially available cytokine combinations.

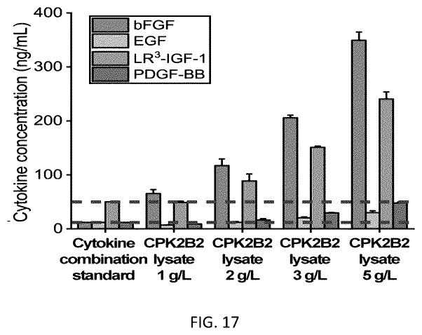

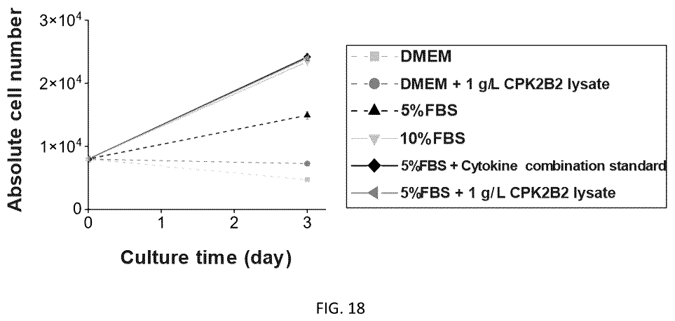

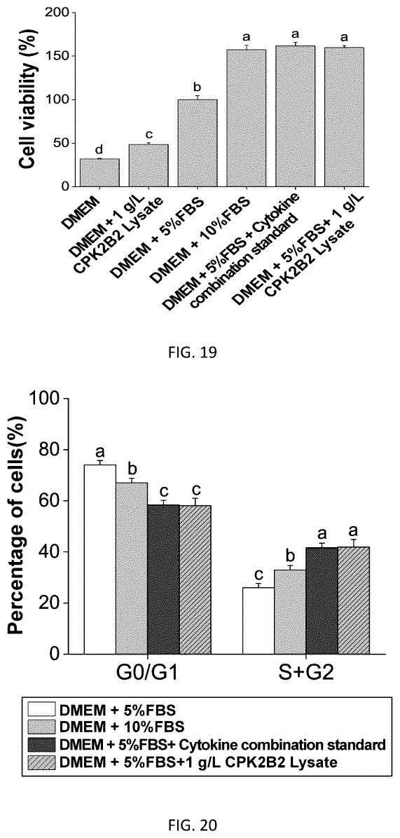

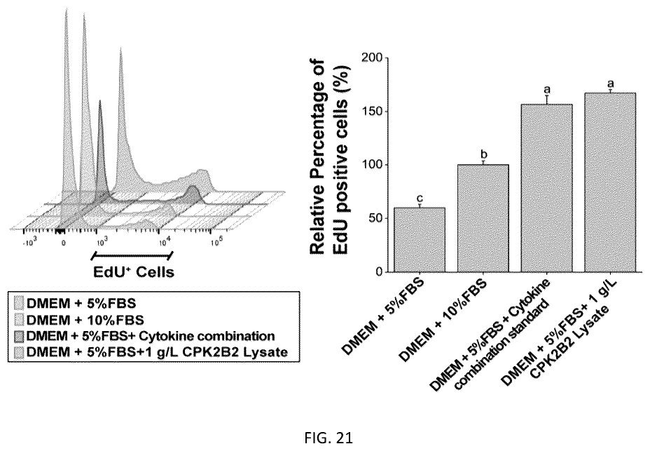

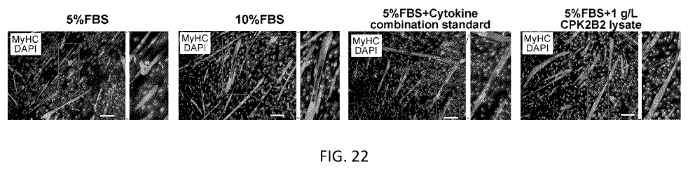

The lysate of recombinant S. cerevisiae CPK2B2 co-expressing the four cytokines was diluted to 1 g/L, 2 g/L, 3 g/L, and 5 g/L, and the yields of the four cytokines were detected by using specific ELISA kits, respectively. The results showed that CPK2B2 lysate could be comparable with a standard sample for commercially available cytokine combination when the concentration of the lysate was 1 g/L (FIG. 17). Muscle stem cells were cultured with DMEM, DMEM+1 g/L CPK2B2 lysate, DMEM+5% FBS, DMEM+10% FBS, DMEM+5% FBS+a standard sample for commercially available cytokine combination, DMEM+5% FBS+1 g/L CPK2B2 lysate, respectively. After 3 d, the cells in each group were counted. The results showed that muscle stem cells could not survive and grow normally in DMEM as basal medium only. On the contrary, the cell survival rate was increased by 16.82% in DMEM supplemented with 1 g/L CPK2B2 lysate, however, the total number of cells after 3 d of culture was still lower than the initial inoculum size. Muscle stem cells could grow normally in DMEM containing 5% FBS and 10% FBS. In addition, the addition of 1 g/L CPK2B2 lysate to DMEM containing 5% FBS significantly promoted cell proliferation, resulting in a 3-fold increase in the number of cells (from 8×10³ to 2.4×10⁴±3.75×10²), which was 1.59 times higher than that of DMEM containing 5% FBS, and not significantly different from DMEM with 10% FBS and DMEM with 5% FBS and a standard sample for commercially available cytokine combination. This indicated that the recombinant cytokine could be comparable with the commercially available cytokine (FIGS. 18-19). The cell cycle and the cells in the active proliferative phase (EdU⁺ cells) of the muscle stem cells cultured under four conditions, namely, DMEM+5% FBS, DMEM+10% FBS, DMEM+5% FBS+a standard sample for commercially available cytokine combination, and DMEM+5% FBS+1 g/L CPK2B2 lysate were investigated, respectively. The results showed that the ratios of G2/S cells and EdU⁺ cells were comparable in the DMEM group containing 5% FBS and 1 g/L CPK2B2 lysate and in the DMEM group containing 5% FBS and a standard sample for commercially available cytokine combination. This indicated that the recombinant cytokine could be comparable with the commercially available cytokine, which significantly promoted the proliferation of muscle stem cells (FIGS. 20-21). During the preparation of cell-cultured meat, muscle stem cells need to be further differentiated to form myotubes and myofibers after massive amplification. Therefore, the differentiation potentials of muscle stem cells after being proliferated in medium supplemented with commercially available cytokines and recombinant cytokines was compared. Firstly, the muscle stem cells were cultured in DMEM+5% FBS, DMEM+10% FBS, DMEM+5% FBS+a standard sample for commercially available cytokine combination and DMEM+5% FBS+1 g/LCPK2B2 lysate for 2 d, respectively. When the cell density reached about 85%, the mediums were replaced with DMEM containing 2% HS, and the differentiation was induced for 4 d. Immunofluorescence assay was carried out to investigate the expression of myosin heavy chain (MyHC) in each group of cells. Immunofluorescence results showed that muscle stem cells proliferated under the above four culture conditions were able to fuse to form multinucleated myotubes and express MyHC, suggesting their myogenic differentiation ability (FIG. 22). This indicated that the recombinant cytokines co-expressed in the CPK2B2 strain could efficiently promote the cell proliferation without affecting their differentiation potential.

### Example 6: Cytokine Costing

The main production strains of commercially available cytokines are E. coli and P. pastoris. The main production processes are as follows: a. obtaining gene fragments of cytokines and constructing recombinant expression strains; b. fermenting the recombinant strains on a large scale and inducing the expression of cytokines; c. enriching and purifying the cytokines by an affinity chromatography, and then subjecting to processes such as elution and desalination to obtain high-purity cytokines; and d. refining to obtain the high-purity cytokines, mainly including removing the endotoxins and freeze-drying, to obtain commercially available finished products. Among them, the cost of cytokine enrichment and purification steps accounts for 50-80% of the total production cost. The method according to the disclosure provides a cytokine production method that can avoid complex downstream purification processes, and can effectively reduce the cost of cytokines for the cell-cultured meat production.

The costs for the cell-cultured meat production of commercially available cytokines from Thermo Fisher Scientific and MCE (MedChem Express) were compared to the recombinant cytokines according to the disclosure by using human-derived cytokines bFGF, EGF and PDGF-BB as examples, respectively. Among them, the cost of the recombinant cytokines according to the disclosure lied mainly in the S. cerevisiae fermentation medium as well as the bacterial fragmentation, endotoxin removal and the like in the downstream process, and the prices of the commercially available cytokines were based on the official prices. bFGF, EGF, and PDGF-BB were applied to cell culture at the concentrations of 10 ng/mL. Specific cost comparisons were shown in Table 4.

TABLE 4
Costing of commercially available cytokines versus recombinant cytokines

| | | | | | Cost for cell- |
|---|---|---|---|---|---|
| | | | Expression | Price | cultured meat |
| Cytokine | Product source | Product name | host | ($/mg) | ($/L) |
| bFGF | Thermo Fisher | Human FGF-basic | E. coli | 1232 | 12.32 |
| | Scientific | (FGF-2/bFGF) (154 aa) | | | |
| | | Human FGF-basic | E. coli | 1848 | 18.48 |
| | | (FGF-2/bFGF) (146 aa) | | | |
| | MCE | FGF2 protein, human | E. coli | 3800 | 38 |
| | | (154 aa) | | | |
| | | FGF basic/bFGF | E. coli | 5000 | 50 |
| | | protein, human (145 aa) | | | |
| | Recombinant | Recombinant bFGF | S. cerevisiae | 2.5 | 0.025 |
| | cytokine | (154 aa) | | | |
| EGF | Thermo Fisher | Human EGF | E. coli | 820 | 8.2 |
| | Scientific | recombinant protein | | | |
| | MCE | EGF protein, human | E. coli | 700 | 7 |
| | Recombinant | Recombinant EGF | S. cerevisiae | 26 | 0.26 |
| | cytokine | | | | |
| PDGF-BB | Thermo Fisher | Human PDGF-BB | E. coli | 5900 | 59 |
| | Scientific | recombinant protein | | | |
| | MCE | PDGF-BB protein, | P. pastoris | 11800 | 118 |
| | | human | | | |
| | Recombinant | Recombinant PDGF-BB | S. cerevisiae | 12 | 0.12 |
| | cytokine | | | | |

The costing results showed that the recombinant cytokine production method provided by the disclosure greatly reduced the production cost of cytokines, reduced the cost in the production of cell-cultured meat to less than USD 0.3 per liter, and could further reduce the cost by further increasing the yield of the recombinantly expressed cytokines, which had the potential for large-scale application and contributed to the rapid development of the cell-cultured meat industry.

Although the disclosure has been disclosed in the above preferred examples, it is not intended to limit the disclosure. Any person familiar with the technology may make various changes and modifications without departing from the spirit and scope according to the disclosure, and therefore the scope of protection according to the disclosure should be based on that defined in the claims.

What is claimed is:

1. A recombinant Saccharomyces cerevisiae, wherein S. cerevisiae is used as a starting strain, the endogenous genes PEP4, YAP3, PRB1 and CYM1 are knocked out, and a cytokine expression cassette is overexpressed;
the cytokine expression cassette comprises cytokine-encoding genes that are combined sequentially or concurrently and whose expression is initiated by a promoter; and
the cytokine-encoding genes comprise a gene encoding basic fibroblast growth factor bFGF; a gene encoding epidermal growth factor EGF; a gene encoding platelet-derived growth factor-AA PDGF-AA; a gene encoding platelet-derived growth factor-BB PDGF-BB; a gene encoding insulin-like growth factor 1 IGF-1; a gene encoding long chain insulin-like growth factor LR³-IGF-1; a gene encoding vascular endothelial growth factor VEGF; a gene encoding hepatocyte growth factor HGF; a gene encoding oncostatin M OSM; a gene encoding interleukin-6 IL-6; and
a gene encoding transforming growth factor-β family TGF-β family.

2. The recombinant S. cerevisiae according to claim 1, wherein the cytokine-encoding genes are genes encoding the cytokines EGF, PDGF-BB, LR³-IGF-1 and bFGF.

3. The recombinant S. cerevisiae according to claim 1, wherein the cytokine-encoding genes are derived from Homo sapiens, Sus scrofa, Bos taurus, Mus musculus, and Rattus norvegicus.

4. The recombinant S. cerevisiae according to claim 2, wherein the expression of the gene encoding the cytokine EGF is initiated by the promoter P_{GAL7}, the expression of the gene encoding the cytokine PDGF-BB is initiated by the promoter P_{FBA1}, the expression of the gene encoding the cytokine LR³-IGF-1 is initiated by the promoter P_{PGK1}, and the expression of the gene encoding the cytokine bFGF is initiated by the promoter P_{ADE2}.

5. The recombinant S. cerevisiae according to claim 1, wherein the overexpression comprises free expression or integrated expression.

6. The recombinant S. cerevisiae according to claim 1, wherein the cytokine expression cassette is integrated into the S. cerevisiae genome at the multi-copy loci Ty1 and Ty2.

7. The recombinant S. cerevisiae according to claim 1, wherein the starting strain is S. cerevisiae C800.

8. A method for preparing a cytokine, comprising:
(1) subjecting the recombinant S. cerevisiae according to claim 1 to a fermentation culture to obtain a bacterial broth; and
(2) physically fragmenting the bacterial broth in a low temperature environment, removing bacterial fragments, removing endotoxins, filtering and sterilizing to obtain the cytokine, wherein the cytokine as prepared is added directly to a medium for cell-cultured meat production without purification.

9. The method according to claim 8, wherein the recombinant S. cerevisiae is inoculated in a liquid YPD medium for fermentation culture.

10. The method according to claim 8, wherein the fermentation culture is carried out in a 3 L fermenter with a rotational speed of 600 rpm, an aeration rate of 3.0 vvm, and a pH of 5.5, when the initial glucose is depleted, a fed-batch fermentation is carried out at a rate of 5 mL/h and terminated after 62 hours, and the fermentation is terminated when the bacterium is no longer growing.

11. Application of the recombinant S. cerevisiae according to claim 1 in the cell-cultured meat production, wherein the recombinant S. cerevisiae is inoculated in a medium for fermentation culture, a lysate of the recombinant S. cerevisiae is obtained at the end of the fermentation, the collected lysate without purification is sterilized, diluted to a certain concentration, added directly to a medium, and applied to the culture of a seed cell of the cell-cultured meat.

12. The application according to claim 11, wherein the seed cell of the cell-cultured meat is a muscle stem cell.

13. The application according to claim 11, wherein the culture of the seed cell of the cell-cultured meat comprises diluting the lysate to 1 g/L, then adding to a DMEM medium containing 5% FBS, filtering, sterilizing, and then applying to the culture of the muscle stem cell.
